(19) United States
(12) Patent Application Publication
(10) Pub. No.: US 2018/0148428 A1
(43) Pub. Date: May 31, 2018

(54) ORGANIC LIGHT EMITTING DEVICE WITH THERMALLY ACTIVATED DELAYED FLUORESCENT LIGHT EMITTING MATERIAL

(71) Applicant: Commonwealth Scientific and Industrial Research Organisation, Acton (AU)

(72) Inventors: James Matthew MacDonald, Victoria (AU); Jenny Lee O'Connell, Victoria (AU); Melissa Skidmore, Victoria (AU); Andrew David Scully, Victoria (AU); Anthony Wilson, Victoria (AU); Kazunori Ueno, Victoria (AU)

(21) Appl. No.: 15/577,511

(22) PCT Filed: May 27, 2016
(86) PCT No.: PCT/AU2016/050421

§ 371 Date: Nov. 28, 2017

(30) Foreign Application Priority Data
May 29, 2015 (AU) 2015902002

(51) Int. Cl. C07D 401/04; C07D 215/06; C07D 215/18; C07D 401/10; C07D 401/14; C07D 413/04; C07D 413/10; C07D 413/14; C07D 417/04; C07D 471/04; C09K 11/06; H01L 51/00

(52) U.S. Cl. CPC C07D 401/04; C07D 215/06; C07D 215/18; C07D 401/10; C07D 401/14; C07D 413/04; H01L 51/0072; C07D 413/14; C07D 417/04; C07D 471/04; C09K 11/06; H01L 51/0061; C07D 413/10

(57) ABSTRACT

The present disclosure is directed to organic electroluminescent devices comprising thermally activated emission material(s) in an emission layer. The present disclosure is also directed to compounds capable of providing thermally activated delayed fluorescence, and to emission layers comprising at least one of the compounds. The compounds may be used in organic layers such as emission layers of an organic electroluminescence device such as fluorescent OLEDs.

### CROSS-REFERENCE TO RELATED APPLICATIONS

The present application claims priority from Australian Provisional Patent Application No 2015902002 filed on 29 May 2015, the content of which is incorporated herein by reference.

### TECHNICAL FIELD

The present disclosure relates to organic electroluminescent devices comprising thermally activated emission material(s) in an emission layer. The present disclosure also relates to compounds capable of providing thermally activated delayed fluorescence, and to emission layers comprising at least one of the compounds.

### BACKGROUND

An organic electroluminescent device is generally comprised of a pair of electrodes forming an anode and a cathode, and one layer or multiple layers comprising a hole injection layer, emission layer (comprised of either fluorescent or phosphorescent material and the host) and electron transporting layer. Into the organic layer(s), holes and electrons are injected from the anode and the cathode respectively, thus resulting in excitons within the emission material. On transition of the excitons to the ground state, the organic luminescence device emits light.

The conventional organic electroluminescent devices were started from a device with a simple structure comprised a layer of an aluminium quinolinol complex (as electron transporting and fluorescent luminescent material) and a layer of a triphenylamine derivative (as a hole transporting material) (Appl. Phys. Lett, vol. 51, pp. 913 (1987)). The maximum internal quantum yield of fluorescent OLEDs has an efficiency limitation of 25%, which was a widely held belief.

More recently, phosphorescent materials have been shown to provide promising dopants for OLEDs by improving the quantum yield of the phosphorescent OLED. The phosphorescent dopant(s) is basically composed of heavy metal complex to induce spin-orbit interaction. In this case a theoretical internal quantum efficiency of 100% is possible (Appl. Phys. Lett., vol. 75, No. 1, pp 4-6 (1999)).

Since then, many examples of efficient phosphorescent materials using the heavy atom have been found. Many of the materials other than those containing an iridium heavy metal have a problem in respect of efficiency and stability. Meanwhile, phosphorescent materials that uses iridium, a relatively rare earth metal, are expensive and have a cost problem from a manufacturing point of view. Moreover, design flexibility in terms of emission colour design is also restricted due to the ligand structure dependence for the complex.

Even more recently, promising advances have been made in the field of fluorescent organic emitting materials. Previously, the maximum internal quantum yield of fluorescent OLEDs was limited to 25% owing to the inability of most organic materials to harvest the statistical 75% of electrically-generated triplet excitons. A new approach to overcome this limitation is to use organic light-emitting materials that have a small S₁-T₁ energy gap. In such materials, thermal excitation of triplet excitons is sufficient to enable efficient reverse intersystem-crossing (RISC) into the singlet state at ambient temperature, thereby allowing a fluorescent organic material to harvest both singlet and triplet excitons. In this case, a proportion of the singlet excitons emit so-called “prompt” fluorescence whereas “delayed” fluorescence from singlet excitons produced upon thermal up-conversion of triplet excitons is relatively long-lived. This phenomenon of enhanced fluorescence is known as thermally-activated delayed fluorescence (TADF) and enables a fluorescent OLED containing such emitting materials to achieve a theoretical internal quantum efficiency of 100% (Appl. Phys. Lett. vol, 101, 093306. (2012)). An alternate type of “delayed” fluorescence is known as triplet-triplet annihilation (TTA). This process occurs when two triplet states collide or interact to produce a singlet excited state and a singlet ground state, leading to “delayed:” fluorescence.

Furthermore, the present disclosure provides, in at least some embodiments, an organic light emitting device comprising an anode, cathode and an intervening organic layer comprising one or more compounds as described herein capable of providing thermally activated delayed fluorescence, which provides alternatives or addresses problems as described above.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY

The present disclosure provides an electroluminescent device comprising a pair of electrodes forming an anode and a cathode, and one layer or multiple layers comprising a hole injection layer, emission layer and electron transporting layer, and wherein the emission layer comprises a light emitting compound as described herein. The compounds as described can be used as organic light emitting materials, and at least in some embodiments as fluorescence emitting materials. The compounds may provide enhanced fluorescence through thermally activated delayed fluorescence (TADF). The compounds may be used in organic layers such as emission layers of an organic electroluminescence device such as fluorescent OLEDs.

In a first aspect, there is provided an electroluminescent device comprising a pair of electrodes forming an anode and a cathode, and one layer or multiple layers comprising a hole injection layer, emission layer and electron transporting layer, and wherein the emission layer comprises a light emitting compound represented by Formula (1):

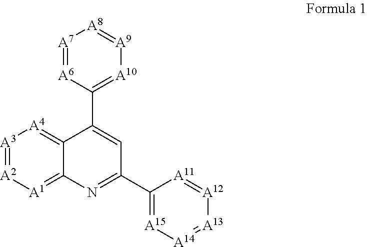

wherein

A¹, A², A³, A⁴, A⁶, A⁷, A⁸, A⁹, A¹⁰, A¹¹, A¹², A¹³, A¹⁴ and A¹⁵, are each independently selected from C—Y and N;

Each Y is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted monocyclic or polycyclic carbocyclic or heterocyclic, a moiety of Formula 2, and where two or more Y groups together form a fused aryl or heteroaryl group optionally substituted with a moiety of Formula 2, wherein the moiety of Formula 2 is represented by:

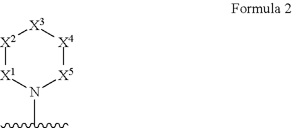

wherein X¹ to X⁵ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic, and two or more R⁴ and R⁵ groups may together form an optionally substituted monocyclic or polycyclic carbocyclic or heterocyclic;

R² and R³are each independently selected from hydrogen, C₁₋₁₀alkyl, and monocyclic or polycyclic aryl and heteroaryl; and

wherein at least one of A¹, A², A³, A⁴, A⁶, A⁷, A⁸, A⁹, A¹⁰, A¹¹, A¹², A¹³, A¹⁴ and A¹⁵, is substituted with the moiety of Formula 2.

In a second aspect, there is provided a thermally activated delayed fluorescence compound of Formula 1:

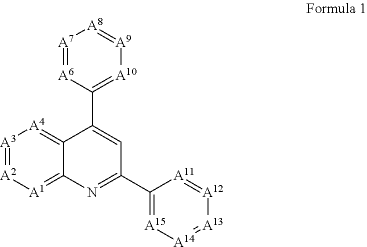

wherein

A¹, A², A³, A⁴, A⁶, A⁷, A⁸, A⁹, A¹⁰, A¹¹, A¹², A¹³, A¹⁴ and A¹⁵, are each independently selected from C—Y and N;

Each Y is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted monocyclic or polycyclic carbocyclic or heterocyclic, a moiety of Formula 2, and where two or more Y groups together form a fused aryl or heteroaryl group optionally substituted with a moiety of Formula 2, wherein the moiety of Formula 2 is represented by:

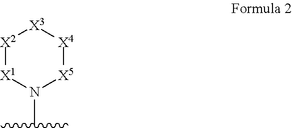

wherein X¹ to X⁵ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic, and two or more R⁴ and R⁵ groups may together form an optionally substituted monocyclic or polycyclic carbocyclic or heterocyclic;

wherein R⁴ and R⁵ cannot both be hydrogen;

R² and R³are each independently selected from hydrogen, C₁₋₁₀alkyl, and monocyclic or polycyclic aryl and heteroaryl; and

wherein at least one of A¹, A², A³, A⁴, A⁶, A⁷, A⁸, A⁹, A¹⁰, A¹¹, A¹², A¹³, A¹⁴ and A¹⁵, is substituted with the moiety of Formula 2.

In relation to the device as described above for the first aspect or the compound as described above for the second aspect, Formula 1 may be provided as further described below or herein.

Formula 1 may be provided wherein each Y is independently selected from hydrogen, deuterium, halo, hydroxyl, nitro, cyano, thiol, amino, optionally substituted C₁₋₆alkyl, a moiety of Formula 2 as described herein, and where two or more Y groups together form a fused aryl or heteroaryl group optionally substituted with a moiety of Formula 2.

Formula 1 may be provided wherein each Y is independently selected from hydrogen, a moiety of Formula 2 as described herein, and where two or more Y groups together form a fused aryl or heteroaryl group optionally substituted with a moiety of Formula 2.

Formula 1 may be provided wherein:

A¹, A², A⁴, A⁶, A⁷, A⁸, A⁹, A¹⁰, A¹¹, A¹², A¹³, A¹⁴ and A¹⁵ are each independently selected from C—Y;

A³ is C—Y, wherein Y is a moiety of Formula 2; and

wherein Y and Formula 2 are as described herein.

Formula 1 may be provided wherein:

A¹, A², A⁴, A⁶, A⁷, A⁸, A⁹, A¹⁰, A¹¹, A¹², A¹³, A¹⁴ and A¹⁵ are each CH; and

A³ is C—Y, wherein Y is a moiety of Formula 2 as described herein.

Formula 1 may be provided wherein:

A¹, A², A³, A⁴, A⁶, A⁷, A⁸, A⁹, A¹⁰, A¹¹, A¹², A¹⁴ and A¹⁵ are each independently selected from C—Y;

A¹³ is C—Y, wherein Y is a moiety of Formula 2; and

wherein Y and Formula 2 are as described herein.

Formula 1 may be provided wherein:

A¹, A², A³, A⁴, A⁶, A⁷, A⁸, A⁹, A¹⁰, A¹¹, A¹², A¹⁴ and A¹⁵ are each CH; and

A¹³ is C—Y, wherein Y is a moiety of Formula 2 as described herein.

Formula 1 may be provided wherein:

A¹, A², A⁴, A⁶, A⁷, A⁸, A⁹, A¹⁰, A¹¹, A¹², A¹⁴ and A¹⁵, are each independently selected from C—Y;

A³ and A¹³ are each C—Y, wherein Y is a moiety of Formula 2; and

wherein Y and Formula 2 are as described herein.

Formula 1 may be provided wherein:

A¹, A², A⁴, A⁶, A⁷, A⁸, A⁹, A¹⁰, A¹¹, A¹², A¹⁴ and A¹⁵, are each CH; and

A³ and A¹³ are each C—Y, wherein Y is a moiety of Formula 2 as described herein.

Formula 1 may be provided wherein:

A¹, A⁶, A⁷, A⁸, A⁹, A¹⁰, A¹¹, A¹², A¹³, A¹⁴ and A¹⁵ are each independently selected from C—Y and N;

A² and A⁴ are each CH;

A³ is C—Y, wherein Y is a moiety of Formula 2; and

wherein Y and Formula 2 are as described herein.

Formula 1 may be provided wherein:

A¹ is CH or N;

A² and A⁴ are each CH;

A³ is C—Y, wherein Y is a moiety of Formula 2 as described herein;

A⁶, A⁷, A⁸, A⁹, A¹⁰, A¹¹, A¹², A¹³, A¹⁴ and A¹⁵, are each independently selected from C—Y, wherein each Y is independently selected from hydrogen, a moiety of Formula 2, and where two or more Y groups together form a fused aryl or heteroaryl group optionally substituted with a moiety of Formula 2.

Formula 2 may be a moiety of Formula 2(a):

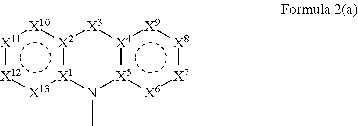

wherein X¹ to X¹³ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, and wherein the dotted circle indicates one or more optional double bonds; and

R² and R³are each independently selected from hydrogen and C₁₋₁₀alkyl.

Formula 2 may be a moiety of Formula 2(a)(i):

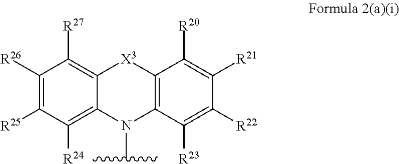

wherein X³ is selected from CR⁴R⁵, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, and optionally substituted aryl or heteroaryl;

R² and R³are each independently selected from hydrogen and C₁₋₁₀alkyl; and

R²⁰ to R²⁷ are each independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, and optionally substituted C₂₋₁₀alkynyl.

Formula 1 may be a compound of Formula 1(a):

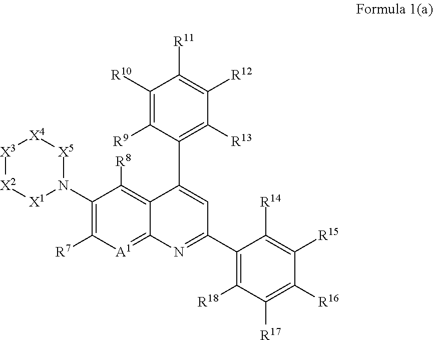

wherein

A¹ is CR⁶ or N;

X¹ to X⁵ are each independently selected from C, CR⁴, CR⁴R⁵, N, NR⁴, S, SiR⁴R⁵ and O, wherein each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic, two or more R⁴ and R⁵ groups may together form an optionally substituted monocyclic or polycyclic carbocyclic or heterocyclic;

Each R² and R³ are independently selected from hydrogen, C₁₋₁₀alkyl, and monocyclic or polycyclic aryl and heteroaryl;

R⁶, R⁷ and R⁸, are each independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl; and

R⁹ to R¹⁸ are each independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic, a moiety of Formula 2 as defined in claim 1, and where two or more groups together form a fused aryl or heteroaryl group optionally substituted with a moiety of Formula 2.

Formula 1 may be a compound of Formula 1(a)(i):

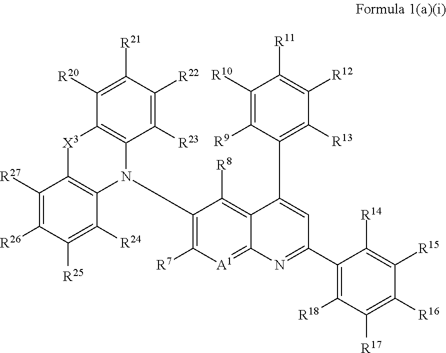

wherein

A¹ is CR⁶ or N,

X³ is selected from CR⁴R⁵, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl;

R² and R³are each independently selected from hydrogen and C₁₋₁₀alkyl; and

R²⁰ to R²⁷ are ach independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl;

R⁶, R⁷ and R⁸, are each independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl;

R⁹ to R¹⁸ are each independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic, a moiety of Formula 2 as defined in claim 1, and where two or more groups together form a fused aryl or heteroaryl group optionally substituted with a moiety of Formula 2.

In a third aspect, there is provided an emission material comprising a compound of Formula 1 or any embodiment thereof as described above or herein.

In a fourth aspect, there is provided a host material for use in an organic electroluminescent device comprising the emission material as described in the third aspect above. The host material may comprise a fluorescent material provided by the compound of Formula 1 and one or more additional fluorescent materials.

In a fifth aspect, there is provided an organic electroluminescent device comprising an anode, a cathode and one or more layers arranged between the anode and the cathode, wherein at least one layer comprises an emission material according to the third aspect above or host material according to the fourth aspect above.

The electroluminescent device according to any of the above aspects or embodiments herein, may comprise one or more layers having a thickness of between 1 nm to 1 μm, 2 nm to 500 nm, or 3 nm to 100 nm, for example 5 to 50 nm. The device may be a stacked organic electroluminescence device for white emission. The device may be a stacked organic electroluminescence device for white emission comprising three emission layers each containing a different emission material. The device may comprise a display. The device may comprise a light source for use in medical applications.

In a sixth aspect, there is provided an imaging agent selected from a compound of Formula 1 or any embodiment thereof as described above or herein. There may be provided a pharmaceutical composition comprising the imaging agent. The imaging agents may comprise any pharmaceutically acceptable salts of the compounds of Formula 1. The imaging agents may be used in diagnostic methods.

In a seventh aspect, there is provided a sensitizer material comprising a compound of Formula 1 or any embodiment thereof as described above or herein. The sensitizer material may be for therapeutic or diagnostic use. There may be provided a pharmaceutical composition comprising the sensitizer material. The sensitizer material may comprise any pharmaceutically acceptable salts of the compounds of Formula 1.

In an eighth aspect, there is provided a polymer comprising a compound of Formula 1 or any embodiment thereof as described above or herein. The compounds of Formula 1 may be incorporated into the polymer. The compounds of Formula 1 may be covalently bonded to the polymer. The compounds of Formula 1 may be used as polymerizable monomers or as pendant groups in polymerizable monomers.

It will be appreciated that in other aspects there may be provided a use, method or process comprising a compound of Formula 1 or any embodiment thereof, as described above or herein.

### BRIEF DESCRIPTION OF DRAWINGS

FIG. 1: PL spectra (a) and PL decay curves (b) for compounds 17-20 films doped in Pyd2Cz (2,6-di(9H-carbazol-9-yl)pyridine)10% w/w (dopant/Pyd2Cz).

FIG. 2: (a) Device structures A and B; (b) HOMO-LUMO energy level diagram of the materials used, energy values are given in eV.

FIG. 3: (a) Luminance-voltage; (b) current-voltage; (c) current efficiency; (d) external quantum efficiency for compounds 17-19.

### DESCRIPTION OF EMBODIMENTS

The present inventors have identified various nitrogen-containing aromatic derivatives that may be used as organic fluorescence emitting materials. The nitrogen-containing aromatic derivatives may provide enhanced fluorescence through thermally activated delayed fluorescence (TADF). The nitrogen-containing aromatic derivatives may be used in organic layers such as emission layers of an organic electroluminescence device such as fluorescent OLEDs.

### Specific Terms

The terms “carbocyclic” and “carbocyclyl” represent a ring system wherein the ring atoms are all carbon atoms, e.g., of about 3 to about 30 carbon atoms, and which may be aromatic, non-aromatic, saturated, or unsaturated, and may be substituted and/or carry fused rings. Examples of such groups include benzene, cyclopentyl, cyclohexyl, or fully or partially hydrogenated phenyl, naphthyl and fluorenyl.

“Heterocyclyl” or “heterocyclic” whether used alone, or in compound words such as heterocyclyloxy represents: (i) an optionally substituted cycloalkyl or cycloalkenyl group, e.g., of about 3 to about 30 ring members, which may contain one or more heteroatoms such as nitrogen, oxygen, or sulfur (examples include pyrrolidinyl, morpholino, thiomorpholino, or fully or partially hydrogenated thienyl, furyl, pyrrolyl, thiazolyl, oxazolyl, oxazinyl, thiazinyl, pyridyl and azepinyl); (ii) an optionally substituted partially saturated polycyclic ring system in which an aryl (or heteroaryl) ring and a heterocyclic group are fused together to form a cyclic structure (examples include phenoxazine, phenothiazine, chromanyl, dihydrobenzofuryl and indolinyl); or (iii) an optionally substituted fully or partially saturated polycyclic fused ring system that has one or more bridges (examples include quinuclidinyl and dihydro-1,4-epoxynaphthyl).

As will be understood, an “aromatic” group means a cyclic group having 4m+2 π electrons, where m is an integer equal to or greater than 1. As used herein, “aromatic” is used interchangeably with “aryl” to refer to an aromatic group, regardless of the valency of aromatic group.

“Aryl” whether used alone, or in compound words such as arylalkyl, aryloxy, aralkyl or arylthio, represents: (i) an optionally substituted mono- or polycyclic aromatic carbocyclic moiety, e.g., of about 6 to about 30 carbon atoms, such as phenyl, naphthyl or fluorenyl; or, (ii) an optionally substituted partially saturated polycyclic carbocyclic aromatic ring system in which an aryl and a cycloalkyl or cycloalkenyl group are fused together to form a cyclic structure such as a tetrahydronaphthyl, indenyl, indanyl or fluorene ring.

A heteroaromatic group is an aromatic group or ring containing one or more heteroatoms, such as N, O, S, Se, Si or P. As used herein, “heteroaromatic” is used interchangeably with “heteroaryl”, and a heteroaryl group refers to monovalent aromatic groups, bivalent aromatic groups and higher multivalency aromatic groups containing one or more heteroatoms.

“Heteroaryl” whether used alone, or in compound words such as heteroaryloxy represents: (i) an optionally substituted mono- or polycyclic aromatic organic moiety, e.g., of about 5 to about 30 ring members in which one or more of the ring members is/are element(s) other than carbon, for example nitrogen, oxygen, sulfur or silicon; the heteroatom(s) interrupting a carbocyclic ring structure and having a sufficient number of delocalized π electrons to provide aromatic character, provided that the rings do not contain adjacent oxygen and/or sulfur atoms. Typical 6-membered heteroaryl groups are pyrazinyl, pyridazinyl, pyrazolyl, pyridyl and pyrimidinyl. All regioisomers are contemplated, e.g., 2-pyridyl, 3-pyridyl and 4-pyridyl. Typical 5-membered heteroaryl rings are furyl, imidazolyl, oxazolyl, isoxazolyl, isothiazolyl, oxadiazolyl, pyrrolyl, 1,3,4-thiadiazolyl, thiazolyl, thienyl, triazolyl, and silole. All regioisomers are contemplated, e.g., 2-thienyl and 3-thienyl. Bicyclic groups typically are benzo-fused ring systems derived from the heteroaryl groups named above, e.g., benzofuryl, benzimidazolyl, benzthiazolyl, indolyl, indolizinyl, isoquinolyl, quinazolinyl, quinolyl and benzothienyl; or, (ii) an optionally substituted partially saturated polycyclic heteroaryl ring system in which a heteroaryl and a cycloalkyl or cycloalkenyl group are fused together to form a cyclic structure such as a tetrahydroquinolyl or pyrindinyl ring.

The term “optionally fused” means that a group is either fused by another ring system or unfused, and “fused” refers to one or more rings that share at least two common ring atoms with one or more other rings. Fusing may be provided by one or more carbocyclic, heterocyclic, aryl or heteroaryl rings, as defined herein, or be provided by substituents of rings being joined together to form a further ring system. The fused ring may be a 5, 6 or 7 membered ring of between 5 and 10 ring atoms in size. The fused ring may be fused to one or more other rings, and may for example contain 1 to 4 rings.

The term “optionally substituted” means that a functional group is either substituted or unsubstituted, at any available position. Substitution can be with one or more functional groups selected from, e.g., alkyl, alkenyl, alkynyl, cycloalkyl, cycloalkenyl, aryl, heterocyclyl, heteroaryl, formyl, alkanoyl, cycloalkanoyl, aroyl, heteroaroyl, carboxyl, alkoxycarbonyl, cycloalkyloxycarbonyl, aryloxycarbonyl, heterocyclyloxycarbonyl, heteroaryloxycarbonyl, alkylaminocarbonyl, cycloalkylaminocarbonyl, arylaminocarbonyl, heterocyclylaminocarbonyl, heteroarylaminocarbonyl, cyano, alkoxy, cycloalkoxy, aryloxy, heterocyclyloxy, heteroaryloxy, alkanoate, cycloalkanoate, aryloate, heterocyclyloate, heteroaryloate, alkylcarbonylamino, cycloalkylcarbonylamino, arylcarbonylamino, heterocyclylcarbonylamino, heteroarylcarbonylamino, nitro, alkylthio, cycloalkylthio, arylthio, heterocyclylthio, heteroarylthio, alkylsulfonyl, cycloalkylsulfonyl, arylsulfonyl, heterocyclysulfonyl, heteroarylsulfonyl, hydroxyl, halo, haloalkyl, haloaryl, haloheterocyclyl, haloheteroaryl, haloalkoxy, haloalkylsulfonyl, silylalkyl, alkenylsilylalkyl, and alkynylsilylalkyl. It will be appreciated that other groups not specifically described may also be used.

The term “halo” or “halogen” whether employed alone or in compound words such as haloalkyl, haloalkoxy or haloalkylsulfonyl, represents fluorine, chlorine, bromine or iodine. Further, when used in compound words such as haloalkyl, haloalkoxy or haloalkylsulfonyl, the alkyl may be partially halogenated or fully substituted with halogen atoms which may be independently the same or different. Examples of haloalkyl include, without limitation, —CH₂CH₂F, —CF₂CF₃ and —CH₂CHFCl. Examples of haloalkoxy include, without limitation, —OCHF₂, —OCF₃, —OCH₂CCl₃, —OCH₂CF₃ and —OCH₂CH₂CF₃. Examples of haloalkylsulfonyl include, without limitation, —SO₂CF₃, —SO₂CCl₃, —SO₂CH₂CF₃ and —SO₂CF₂CF₃.

“Alkyl” whether used alone, or in compound words such as alkoxy, alkylthio, alkylamino, dialkylamino or haloalkyl, represents straight or branched chain hydrocarbons ranging in size from one to about 20 carbon atoms, or more. Thus alkyl moieties include, unless explicitly limited to smaller groups, moieties ranging in size, for example, from one to about 6 carbon atoms or greater, such as, methyl, ethyl, n-propyl, iso-propyl and/or butyl, pentyl, hexyl, and higher isomers, including, e.g., those straight or branched chain hydrocarbons ranging in size from about 6 to about 20 carbon atoms, or greater.

“Alkenyl” whether used alone, or in compound words such as alkenyloxy or haloalkenyl, represents straight or branched chain hydrocarbons containing at least one carbon-carbon double bond, including, unless explicitly limited to smaller groups, moieties ranging in size from two to about 6 carbon atoms or greater, such as, methylene, ethylene, 1-propenyl, 2-propenyl, and/or butenyl, pentenyl, hexenyl, and higher isomers, including, e.g., those straight or branched chain hydrocarbons ranging in size, for example, from about 6 to about 20 carbon atoms, or greater.

“Alkynyl” whether used alone, or in compound words such as alkynyloxy, represents straight or branched chain hydrocarbons containing at least one carbon-carbon triple bond, including, unless explicitly limited to smaller groups, moieties ranging in size from, e.g., two to about 6 carbon atoms or greater, such as, ethynyl, 1-propynyl, 2-propynyl, and/or butynyl, pentynyl, hexynyl, and higher isomers, including, e.g., those straight or branched chain hydrocarbons ranging in size from, e.g., about 6 to about 20 carbon atoms, or greater.

“Cycloalkyl” represents a mono- or polycarbocyclic ring system of varying sizes, e.g., from about 3 to about 20 carbon atoms, e.g., cyclopropyl, cyclobutyl, cyclopentyl, cyclohexyl or cycloheptyl. The term cycloalkyloxy represents the same groups linked through an oxygen atom such as cyclopentyloxy and cyclohexyloxy. The term cycloalkylthio represents the same groups linked through a sulfur atom such as cyclopentylthio and cyclohexylthio.

“Cycloalkenyl” represents a non-aromatic mono- or polycarbocyclic ring system, e.g., of about 3 to about 20 carbon atoms containing at least one carbon-carbon double bond, e.g., cyclopentenyl, cyclohexenyl or cycloheptenyl. The term “cycloalkenyloxy” represents the same groups linked through an oxygen atom such as cyclopentenyloxy and cyclohexenyloxy. The term “cycloalkenylthio” represents the same groups linked through a sulfur atom such as cyclopentenylthio and cyclohexenylthio.

“Cycloalkynyl” represents a non-aromatic mono- or polycarbocyclic ring system, e.g., of about 3 to about 20 carbon atoms containing at least one carbon-carbon double bond, e.g., cyclopentenyl, cyclohexenyl or cycloheptenyl. The term “cycloalkenyloxy” represents the same groups linked through an oxygen atom such as cyclopentenyloxy and cyclohexenyloxy. The term “cycloalkenylthio” represents the same groups linked through a sulfur atom such as cyclopentenylthio and cyclohexenylthio.

“Formyl” represents a —CHO moiety.

“Allcanoyl” represents a —C(═O)-alkyl group in which the alkyl group is as defined supra. In a particular embodiment, an alkanoyl ranges in size from about C₂-C₂₀. One example is acyl.

“Aroyl” represents a —C(═O)-aryl group in which the aryl group is as defined supra. In a particular embodiment, an aroyl ranges in size from about C₇-C₂₀. Examples include benzoyl and 1-naphthoyl and 2-naphthoyl.

“Heterocycloyl” represents a —C(═O)-heterocyclyl group in which the heterocylic group is as defined supra. In a particular embodiment, an heterocycloyl ranges in size from about C₄-C₂₀.

“Heteroaroyl” represents a —C(═O)-heteroaryl group in which the heteroaryl group is as defined supra. In a particular embodiment, a heteroaroyl ranges in size from about C₆-C₂₀. An example is pyridylcarbonyl.

“Carboxyl” represents a —CO₂H moiety.

“Oxycarbonyl” represents a carboxylic acid ester group —CO₂R which is linked to the rest of the molecule through a carbon atom.

“Allcoxycarbonyl” represents an —CO₂-alkyl group in which the alkyl group is as defined supra. In a particular embodiment, an alkoxycarbonyl ranges in size from about C₂-C₂₀. Examples include methoxycarbonyl and ethoxycarbonyl.

“Aryloxycarbonyl” represents an —CO₂-aryl group in which the aryl group is as defined supra. Examples include phenoxycarbonyl and naphthoxycarbonyl.

“Heterocyclyloxycarbonyl” represents a —CO₂-heterocyclyl group in which the heterocyclic group is as defined supra.

“Heteroaryloxycarbonyl” represents a —CO-heteroaryl group in which the heteroaryl group is as defined supra.

“Aminocarbonyl” represents a carboxylic acid amide group —C(═O)NHR or —C(═O)NR₂ which is linked to the rest of the molecule through a carbon atom.

“Allcylaminocarbonyl” represents a —C(═O)NHR or —C(═O)NR₂ group in which R is an alkyl group as defined supra.

“Arylaminocarbonyl” represents a —C(═O)NHR or —C(═O)NR₂ group in which R is an aryl group as defined supra.

“Heterocyclylaminocarbonyl” represents a —C(═O)NHR or —C(═O)NR₂ group in which R is a heterocyclic group as defined supra. In certain embodiments, NR₂ is a heterocyclic ring, which is optionally substituted.

“Heteroarylaminocarbonyl” represents a —C(═O)NHR or —C(═O)NR₂ group in which R is a heteroaryl group as defined supra. In certain embodiments, NR₂ is a heteroaryl ring, which is optionally substituted.

“Cyano” represents a —CN moiety.

“Hydroxyl” represents a —OH moiety.

“Allcoxy” represents an —O-alkyl group in which the alkyl group is as defined supra. Examples include methoxy, ethoxy, n-propoxy, iso-propoxy, and the different butoxy, pentoxy, hexyloxy and higher isomers.

“Aryloxy” represents an —O-aryl group in which the aryl group is as defined supra. Examples include, without limitation, phenoxy and naphthoxy.

“Alkenyloxy” represents an —O-alkenyl group in which the alkenyl group is as defined supra. An example is allyloxy.

“Heterocyclyloxy” represents an —O-heterocyclyl group in which the heterocyclic group is as defined supra.

“Heteroaryloxy” represents an —O-heteroaryl group in which the heteroaryl group is as defined supra. An example is pyridyloxy.

“Alkanoate” represents an —OC(═O)—R group in which R is an alkyl group as defined supra.

“Aryloate” represents a —OC(═O)—R group in which R is an aryl group as defined supra.

“Heterocyclyloate” represents an —OC(═O)—R group in which R is a heterocyclic group as defined supra.

“Heteroaryloate” represents an —OC(═O)—R group in which P is a heteroaryl group as defined supra.

“Amino” represents an —NH₂ moiety.

“Alkylamino” represents an —NHR or —NR₂ group in which R is an alkyl group as defined supra. Examples include, without limitation, methylamino, ethylamino, n-propylamino, isopropylamino, and the different butylamino, pentylamino, hexylamino and higher isomers.

“Arylamino” represents an —NHR or —NR₂ group in which R is an aryl group as defined supra. An example is phenylamino.

“Heterocyclylamino” represents an —NHR or —NR₂ group in which R is a heterocyclic group as defined supra. In certain embodiments, NR₂ is a heterocyclic ring, which is optionally substituted.

“Heteroarylamino” represents a —NHR or —NR₂ group in which R is a heteroaryl group as defined supra. In certain embodiments, NR₂ is a heteroaryl ring, which is optionally substituted.

“Carbonylamino” represents a carboxylic acid amide group —NHC(═O)R that is linked to the rest of the molecule through a nitrogen atom.

“Alkylcarbonylamino” represents a —NHC(═O)R group in which R is an alkyl group as defined supra.

“Arylcarbonylamino” represents an —NHC(═O)R group in which R is an aryl group as defined supra.

“Heterocyclylcarbonylamino” represents an —NHC(═O)R group in which R is a heterocyclic group as defined supra.

“Heteroarylcarbonylamino” represents an —NHC(═O)R group in which R is a heteroaryl group as defined supra.

“Nitro” represents a —NO₂ moiety.

“Alkylthio” represents an —S-alkyl group in which the alkyl group is as defined supra. Examples include, without limitation, methylthio, ethylthio, n-propylthio, iso propylthio, and the different butylthio, pentylthio, hexylthio and higher isomers.

“Arylthio” represents an —S-aryl group in which the aryl group is as defined supra. Examples include phenylthio and naphthylthio.

“Heterocyclylthio” represents an —S-heterocyclyl group in which the heterocyclic group is as defined supra.

“Heteroarylthio” represents an —S-heteroaryl group in which the heteroaryl group is as defined supra.

“Sulfonyl” represents an —SO₂R group that is linked to the rest of the molecule through a sulfur atom.

“Alkylsulfonyl” represents an —SO₂-alkyl group in which the alkyl group is as defined supra.

“Arylsulfonyl” represents an —SO₂-aryl group in which the aryl group is as defined supra.

“Heterocyclylsulfonyl” represents an —SO₂-heterocyclyl group in which the heterocyclic group is as defined supra.

“Heteoarylsulfonyl” presents an —SO₂-heteroaryl group in which the heteroaryl group is as defined supra.

“Aldehyde” represents a —C(═O)H group.

“Alkanal” represents an alkyl-(C═O)H group in which the alkyl group is as defined supra.

“Alkylsilyl” presents an alkyl group that is linked to the rest of the molecule through the silicon atom, which may be substituted with up to three independently selected alkyl groups in which each alkyl group is as defined supra.

“Alkenylsilyl” presents an alkenyl group that is linked to the rest of the molecule through the silicon atom, which may be substituted with up to three independently selected alkenyl groups in which each alkenyl group is as defined supra.

“Alkynylsilyl” presents an alkynyl group that is linked to the rest of the molecule through the silicon atom, which may be substituted with up to three independently selected alkynyl groups in which each alkenyl group is as defined supra.

### General Terms

“Pharmaceutically acceptable salt” or “salts thereof” means organic or inorganic salts of the pharmaceutically important molecule. A pharmaceutically acceptable salt may involve the inclusion of another molecule such as an acetate ion, a succinate ion or other counterion. The counterion may be any organic or inorganic moiety that stabilizes the charge on the parent compound. For example, a salt may be provided by reaction of a tertiary amine group to form a quaternary ammonium group having a counterion, such as n-butylammonium bromide. Furthermore, a pharmaceutically important organic molecule may have more than one charged atom in its structure. Situations where multiple charged atoms are part of the molecule may have multiple counterions. Hence, the molecule of a pharmaceutically acceptable salt may contain one or more than one charged atom and may also contain one or more than one counterion. The desired charge distribution is determined according to methods of drug administration. Examples of pharmaceutically acceptable salts are well known in the art but, without limiting the scope of the present invention, exemplary presentations can be found in the Physician's Desk Reference, The Merck Index, The Pharmacopoeia and Goodman & Gilman's The Pharmacological Basis of Therapeutics.

“Treat,” “treating,” or “treatment” as used herein refers to any type of measure that imparts a benefit to a patient afflicted with or at risk for developing a disease, including improvement in the condition of the patient (e.g., in one or more symptoms), delay in the onset or progression of the disease, etc. Treatment may include any drug, drug product, method, procedure, lifestyle change, or other adjustment introduced in attempt to effect a change in a particular aspect of a subject's health (i.e., directed to a particular disease, disorder, or condition).

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be clearly understood that, although a number of prior art publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Thermally Activated Delayed Fluorescence and Triplet-Triplet Annihilation Compounds

The present disclosure provides thermally activated delayed fluorescence and triplet-triplet annihilation compounds of Formula 1 as described below. The compounds may be suitable for use as emission materials or within emission layers of electronic devices, such as fluorescent OLEDs.

The compounds of Formula 1 may be a compound according to the following structure:

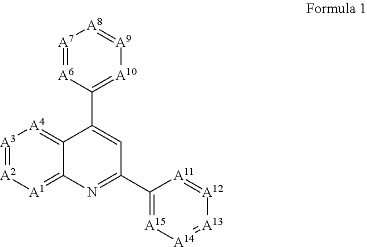

wherein

A¹, A², A³, A⁴, A⁶, A⁷, A⁸, A⁹, A¹⁰, A¹¹, A¹², A¹³, A¹⁴ and A¹⁵, are each independently selected from C—Y and N;

Each Y is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted monocyclic or polycyclic carbocyclic or heterocyclic, a moiety of Formula 2, and where two or more Y groups together form a fused aryl or heteroaryl group optionally substituted with a moiety of Formula 2, wherein the moiety of Formula 2 is represented by:

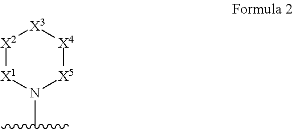

wherein X¹ to X⁵ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic, and two or more R⁴ and R⁵ groups may together form an optionally substituted monocyclic or polycyclic carbocyclic or heterocyclic;

R² and R³are each independently selected from hydrogen, C₁₋₁₀alkyl, and monocyclic or polycyclic aryl and heteroaryl; and

wherein at least one of A¹, A², A³, A⁴, A⁶, A⁷, A⁸, A⁹, A¹⁰, A¹¹, A¹², A¹³, A¹⁴ and A¹⁵, is substituted with the moiety of Formula 2.

The compounds of Formula 1 may be a compound according to the following structure:

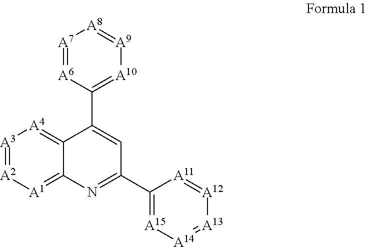

wherein

A¹, A², A³, A⁴, A⁶, A⁷, A⁸, A⁹, A¹⁰, A¹¹, A¹², A¹³, A¹⁴ and A¹⁵, are each independently selected from C—Y and N;

Each Y is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted monocyclic or polycyclic carbocyclic or heterocyclic, a moiety of Formula 2, and where two or more Y groups together form a fused aryl or heteroaryl group optionally substituted with a moiety of Formula 2, wherein the moiety of Formula 2 is represented by:

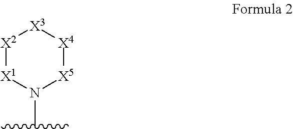

wherein X¹ to X⁵ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic, and two or more R⁴ and R⁵ groups may together form an optionally substituted monocyclic or polycyclic carbocyclic or heterocyclic;

wherein R⁴ and R⁵ cannot both be hydrogen;

R² and R³are each independently selected from hydrogen, C₁₋₁₀alkyl, and monocyclic or polycyclic aryl and heteroaryl; and wherein at least one of A¹, A², A³, A⁴, A⁶, A⁷, A⁸, A⁹, A¹⁰, A¹¹, A², A¹³, A¹⁴ and A¹⁵, is substituted with the moiety of Formula 2.

The compounds of Formula 1 may be a compound according to the following structure:

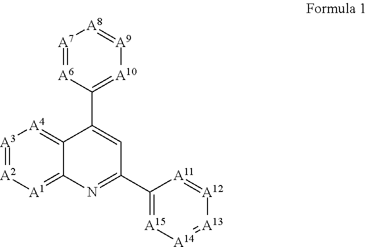

wherein

A¹, A², A³, A⁴, A⁶, A⁷, A⁸, A⁹, A¹⁰, A¹¹, A¹², A¹³, A¹⁴ and A¹⁵, are each independently selected from C—Y and N;

Each Y is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted monocyclic or polycyclic carbocyclic or heterocyclic, a moiety of Formula 2, and where two or more Y groups together form a fused aryl or heteroaryl group optionally substituted with a moiety of Formula 2, wherein the moiety of Formula 2 is represented by:

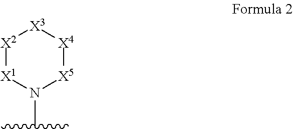

wherein X¹ to X⁵ are each independently selected from C, CR⁴, CR⁴R⁵, N, NR⁴, S, SO, SO₂, SiR⁴R⁵ and O;

R⁴ is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic;

R⁵ is independently selected from deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic; and

two or more R⁴ and R⁵ groups may together form an optionally substituted monocyclic or polycyclic carbocyclic or heterocyclic;

R² and R³are each independently selected from hydrogen, C₁₋₁₀alkyl, and monocyclic or polycyclic aryl and heteroaryl; and

wherein at least one of A¹, A², A³, A⁴, A⁶, A⁷, A⁸, A⁹, A¹⁰, A¹¹, A¹², A¹³, A¹⁴ and A¹⁵, is substituted with the moiety of Formula 2.

The compounds of Formula 1 may be a compound according to the following structure:

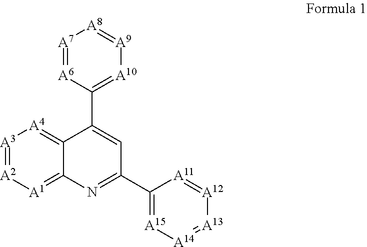

wherein

A¹, A², A³, A⁴, A⁶, A⁷, A⁸, A⁹, A¹⁰, A¹¹, A¹², A¹³, A¹⁴ and A¹⁵, are each independently selected from C—Y and N;

Each Y is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted monocyclic or polycyclic carbocyclic or heterocyclic, a moiety of Formula 2, and where two or more Y groups together form a fused aryl or heteroaryl group optionally substituted with a moiety of Formula 2, wherein the moiety of Formula 2 is represented by:

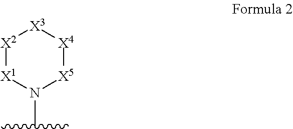

wherein X¹ to X⁵ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, an optionally substituted monocyclic or polycyclic heterocyclic, and two or more R⁴ and R⁵ groups may together form an optionally substituted monocyclic or polycyclic carbocyclic or heterocyclic;

R² and R³are each independently selected from hydrogen, C₁₋₁₀alkyl, and monocyclic or polycyclic aryl and heteroaryl;

wherein at least one of A¹, A², A³, A⁴, A⁶, A⁷, A⁸, A⁹, A¹⁰, A¹¹, A¹², A¹³, A¹⁴ and A¹⁵, is substituted with the moiety of Formula 2;

with the proviso that the compound of Formula 1 is not:

(2-(5-chloro-2-fluorophenyl)-4-[8-(1-piperazinyl)-5-isoquinolinyl]-1,8-naphthyridine);

5-amino-7-(4-morpholinyl)-2,4-diphenyl-1,6-Naphthyridine-8-carbonitrile;

5-(3-bromophenyl)-7-[4-(4-morpholinyl)phenyl]-pyrido[2,3-d]pyrimidin-4-amine;

5-(3-bromophenyl)-7-[6-(4-morpholinyl)-3-pyridinyl]-pyrido[2,3-d]pyrimidin-4-amine;

5-(3-bromophenyl)-7-[2-(4-morpholinyl)-5-pyrimidinyl]-pyrido[2,3-d]pyrimidin-4-amine;

5-(3-bromophenyl)-7-[2-(4-morpholinyl)-5-pyrimidinyl]-pyrido[2,3-d]pyrimidin-4-amine hydrochloride (5:6);

5-(3-bromophenyl)-7-[6-(4-morpholinyl)-3-pyridazinyl]-pyrido[2,3-d]pyrimidin-4-amine;

5-(3-bromophenyl)-7-[5-(4-morpholinyl)-2-pyridinyl]-pyrido[2,3-d]pyrimidin-4-amine;

5-(3-bromophenyl)-7-[5-(4-morpholinyl)-2-pyridinyl]-pyrido[2,3-d]pyrimidin-4-amine hydrochloride (1:3);

5-(3-bromophenyl)-7-[5-(4-morpholinyl)-2-pyrazinyl]-pyrido[2,3-d]pyrimidin-4-amine;

5-(3-bromophenyl)-7-[5-(4-morpholinyl)-2-pyrazinyl]-pyrido[2,3-d]pyrimidin-4-amine hydrochloride (17:25);

5-(3-bromophenyl)-7-[6-(4-ethoxy-1-piperidinyl)-3-pyridazinyl]-pyrido[2,3-d]pyrimidin-4-amine;

5-(3-bromophenyl)-7-[6-[4-[2-[(tetrahydro-2H-pyran-4-yl)oxy]ethyl]-1-piperidinyl]-3-pyridazinyl]-pyrido[2,3-d]pyrimidin-4-amine;

3-[1-[5-[4-amino-5-(3-bromophenyl)pyrido[2,3-d]pyrimidin-7-yl]-2-pyridinyl]-4-hydroxy-4-piperidinyl]dihydro-2(3H)-furanone;

5-(3-bromophenyl)-7-[6-(1,3-dioxa-8-azaspiro[4.5]dec-8-yl)-3-pyridinyl]-pyrido[2,3-d]pyrimidin-4-amine;

5-(3-bromophenyl)-7-[6-(1,3-dioxa-8-azaspiro[4.5]dec-8-yl)-3-pyridinyl]-pyrido[2,3-d]pyrimidin-4-amine;

5-(3-bromophenyl)-7-[6-[4-[[(tetrahydro-2H-pyran-4-yl)oxy]methyl]-1-piperidinyl]-3-pyridazinyl]-pyrido[2,3-d]pyrimidin-4-amine;

5-(3-bromophenyl)-7-[6-(2-oxa-5-azabicyclo[2.2.1]hept-5-yl)-3-pyridinyl]-pyrido[2,3-d]pyrimidin-4-amine;

5-(3-bromophenyl)-7-[6-(1,4-dioxa-8-azaspiro[4.5]dec-8-yl)-3-pyridinyl]-pyrido[2,3-d]pyrimidin-4-amine;

5-(3-bromophenyl)-7-[6-(4-thiomorpholinyl)-3-pyridinyl]-pyrido[2,3-d]pyrimidin-4-amine;

1-[5-[4-amino-5-(3-bromophenyl)pyrido[2,3-d]pyrimidin-7-yl]-2-pyridinyl]-4-piperidinol;

5-(2-bromophenyl)-7-[6-(4-morpholinyl)-3-pyridinyl]-pyrido[2,3-d]pyrimidin-4-amine;

5-(4-bromophenyl)-7-[6-(4-morpholinyl)-3-pyridinyl]-pyrido[2,3-d]pyrimidin-4-amine; 5-(3-chlorophenyl)-7-[6-(4-morpholinyl)-3-pyridinyl]-pyrido[2,3-d]pyrimidin-4-amine;

5-(2,3-dichlorophenyl)-7-[6-(4-morpholinyl)-3-pyridinyl]-pyrido[2,3-d]pyrimidin-4-amine;

5-(2,5-dichlorophenyl)-7-[6-(4-morpholinyl)-3-pyridinyl]-pyrido[2,3-d]pyrimidin-4-amine;

7-[6-(4-morpholinyl)-3-pyridinyl]-5-(3-pyridinyl)-pyrido[2,3-d]pyrimidin-4-amine;

5-(3-bromophenyl)-7-[6-[(3S)-3-[(tetrahydro-2-furanyl)oxy]-1-piperidinyl]-3-pyridazinyl]-pyrido[2,3-d]pyrimidin-4-amine;

2-[4-(4-morpholinyl)phenyl]-4-phenyl-quinoline;

5-(3-bromophenyl)-7-[4-(3-fluoro-4-thiomorpholinyl)phenyl]-pyrido[2,3-d]pyrimidin-4-amine;

5-amino-4-(4-methoxyphenyl)-2-phenyl-7-(1-piperidinyl)-1,6-naphthyridine-8-carbonitrile;

5-(3-bromophenyl)-7-[6-[(3R)-3-[(tetrahydro-2-furanyl)oxy]-1-piperidinyl]-3-pyridazinyl]-pyrido[2,3-d]pyrimidin-4-amine;

8-[6-[4-amino-5-(3-bromophenyl)pyrido[2,3-d]pyrimidin-7-yl]-3-pyridazinyl]-1-ox -8-azaspiro[4.5]decane-3,4-dione;

5-(3-bromophenyl)-7-[6-(1,5-dioxa-9-azaspiro[5.5]undec-9-yl)-3-pyridazinyl]-pyrido[2,3-d]pyrimidin-4-amine;

4-[1-[6-[4-amino-5-(3-bromophenyl)pyrido[2,3-d]pyrimidin-7-yl]-3-pyridazinyl]-4-hydroxy-4-piperidinyl]dihydro-2(3H)-furanone;

4-[1-[5-[4-amino-5-(3-bromophenyl)pyrido[2,3-d]pyrimidin-7-yl]-2-pyridinyl]-4-hydroxy-4-piperidinyl]-2(5H)-furanone;

5-[4-amino-5-(3-bromophenyl)pyrido[2,3-d]pyrimidin-7-yl]-2-[4-(hydroxymethyl)-1-piperidinyl]-4-pyridinol;

5-(3-bromophenyl)-7-[6-[(2R,6S)-2,6-dimethyl-4-morpholinyl]-3-pyridinyl]-pyrido[2,3-d]pyrimidin-4-amine;

8-[6-[4-amino-5-(3-bromophenyl)pyrido[2,3-d]pyrimidin-7-yl]-3-pyridazinyl]-8-azaspiro[4.5]decane-1,2-dione;

5-(3-bromophenyl)-7-[6-(4-morpholinyl)-2-pyridinyl]-pyrido[2,3-d]pyrimidin-4-amine; and

5-(3-chlorophenyl)-7-[6-(1,4-dioxa-8-azaspiro[4.5]dec-8-0-3-pyridinyl] pyrido[2,3-d]pyrimidin-4-amine.

A compound of Formula 1 may be provided wherein:

A¹, A², A⁴, A⁶, A⁷, A⁸, A⁹, A¹⁰, A¹¹, A¹², A¹³, A¹⁴ and A¹⁵, are each independently selected from C—Y and N, wherein Y is any embodiment of Y as described herein; and

A³ is C—Y, wherein Y is a moiety of Formula 2, wherein Formula 2 may be any embodiment of Formula 2 as described herein.

A compound of Formula 1 may be provided wherein:

A¹, A⁶, A⁷, A⁸, A⁹, A¹⁰, A¹¹, A¹², A¹³, A¹⁴ and A¹⁵ are each independently selected from C—Y and N, wherein Y is any embodiment of Y as described herein;

A² and A⁴ are each CH;

A³ is C—Y, wherein Y is a moiety of Formula 2, and wherein Formula 2 is any embodiment of Formula 2 as described herein.

A compound of Formula 1 may be provided wherein:

A¹ is CH or N;

A² and A⁴ are each CH;

A³ is C—Y, wherein Y is a moiety of Formula 2; wherein Formula 2 is any embodiment of Formula 2 as described herein;

A⁶, A⁷, A⁸, A⁹, A¹⁰, A¹¹, A¹², A¹³, A¹⁴ and A¹⁵ are each independently selected from C—Y, wherein Y is any embodiment of Y as described herein.

A compound of Formula 1 may be provided wherein:

A¹ is CH or N;

A² and A⁴ are each CH;

A³ is C—Y, wherein Y is a moiety of Formula 2 as described herein;

A⁶, A⁷, A⁸, A⁹, A¹⁰, A¹¹, A¹², A¹⁴ and A¹⁵, are each independently selected from C—Y, wherein Y is as described herein; and

A¹³ is selected from C—Y; wherein Y is selected from a moiety of Formula 2 or where two or more Y groups together form a fused aryl or heteroaryl group optionally substituted with a moiety of Formula 2.

A compound of Formula 1 may be provided wherein:

A¹ is CH or N;

A², A⁴, A⁶, A⁷, A⁸, A⁹, A¹⁰, A¹¹, A¹², A¹³, A¹⁴ and A¹⁵, are each CH;

A³ is C—Y, wherein Y is a moiety of Formula 2, and wherein Formula 2 may be any embodiment of Formula 2 as described herein.

A compound of Formula 1 may be provided wherein:

A¹, A², A⁴, A⁶, A⁷, A⁸, A⁹, A¹⁰, A¹¹, A¹², A¹³, A¹⁴ and A¹⁵ are each CH; and

A³ is C—Y, wherein Y is a moiety of Formula 2, and wherein Formula 2 may be any embodiment of Formula 2 as described herein.

The moiety of Formula 2 as described for any of the above embodiments may be a moiety of Formula 2(a):

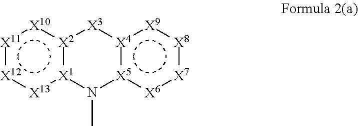

wherein X¹ to X¹³ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, and wherein the dotted circle indicates one or more optional double bonds; and

R² and R³are each independently selected from hydrogen and C₁₋₁₀alkyl.

The moiety of Formula 2 as described for any of the above embodiments may be a moiety of Formula 2(a)(i):

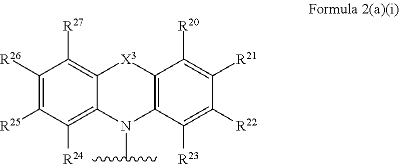

wherein X³ is selected from CR⁴R⁵, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl;

R² and R³are each independently selected from hydrogen and C₁₋₁₀alkyl; and

R²⁰ to R²⁷ are ach independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl.

The compound of Formula 2 may be, for example, selected from phenoxazine, phenothiazine and acridine.

A compound of Formula 1 may be provided wherein:

A¹, A², A³, A⁴, A⁶, A⁷, A⁸, A⁹, A¹⁰, A¹¹, A¹², A¹³, A¹⁴ and A¹⁵, are each independently selected from any of the above embodiments; and

Each Y is independently selected from hydrogen, deuterium, halo, hydroxyl, nitro, cyano, thiol, amino, optionally substituted C₁₋₆alkyl, optionally substituted tricyclic carbocyclic or heterocyclic, where two or more Y groups together form a fused phenyl group optionally substituted with a moiety of Formula 2 as described herein.

It will be appreciated that in other aspects there may be provided a use, method or process comprising a compound of Formula 1 or any embodiment thereof, as described above or herein.

The compounds, or compositions thereof, described herein may include salts, solvates, hydrates, isomers, tautomers, racemates, stereoisomers, enantiomers or diastereoisomers of those compounds.

Some specific examples of compounds represented by Formula 1 may include the following non-limiting examples:

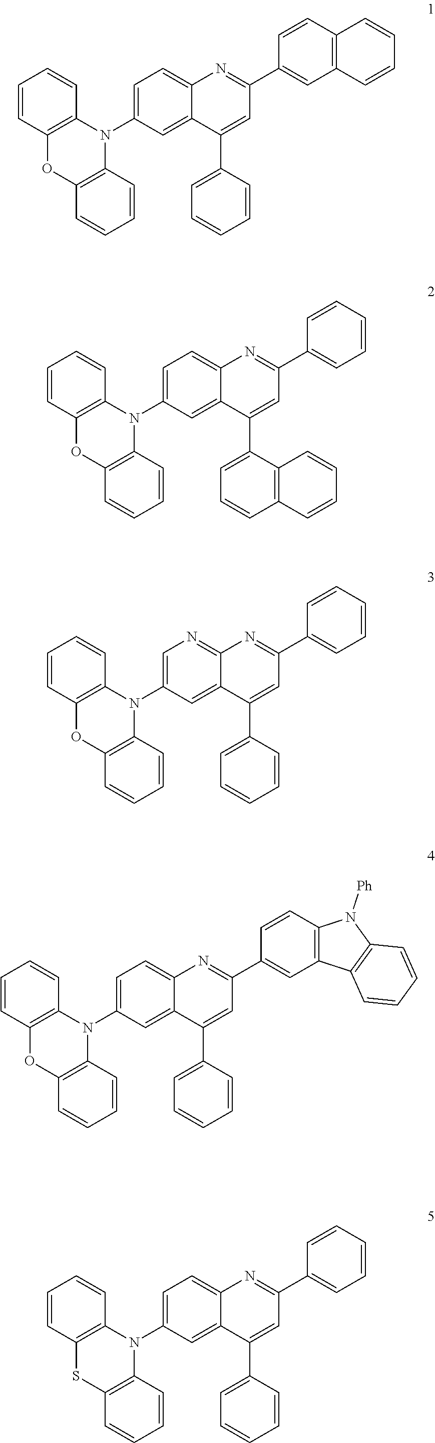 [SMILES: c1ccc(-c2cc(-c3ccc4ccccc4c3)nc3ccc(N4c5ccccc5Oc5ccccc54)cc23)cc1 ; c1ccc(-c2cc(-c3cccc4ccccc34)c3cc(N4c5ccccc5Oc5ccccc54)ccc3n2)cc1 ; c1ccc(-c2cc(-c3ccccc3)c3cc(N4c5ccccc5Oc5ccccc54)cnc3n2)cc1 ; c1ccc(-c2cc(-c3ccc4c(c3)c3ccccc3n4-c3ccccc3)nc3ccc(N4c5ccccc5Oc5ccccc54)cc23)cc1 ; c1ccc(-c2cc(-c3ccccc3)c3cc(N4c5ccccc5Sc5ccccc54)ccc3n2)cc1]

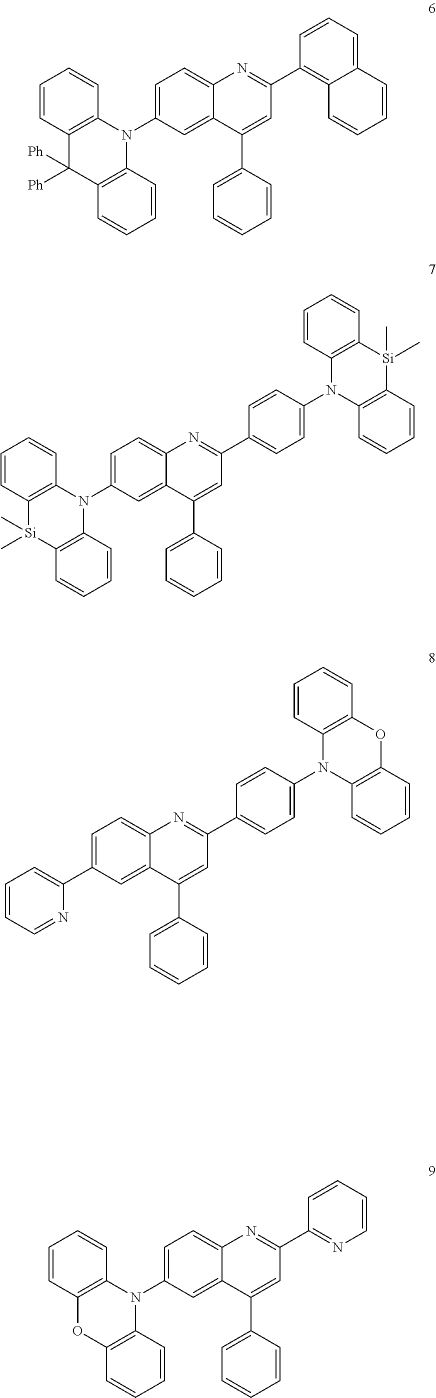 [SMILES: c1ccc(-c2cc(-c3cccc4ccccc34)nc3ccc(N4c5ccccc5C(c5ccccc5)(c5ccccc5)c5ccccc54)cc23)cc1 ; C[Si]1(C)c2ccccc2N(c2ccc(-c3cc(-c4ccccc4)c4cc(N5c6ccccc6[Si](C)(C)c6ccccc65)ccc4n3)cc2)c2ccccc21 ; c1ccc(-c2cc(-c3ccc(N4c5ccccc5Oc5ccccc54)cc3)nc3ccc(-c4ccccn4)cc23)cc1 ; c1ccc(-c2cc(-c3ccccn3)nc3ccc(N4c5ccccc5Oc5ccccc54)cc23)cc1]

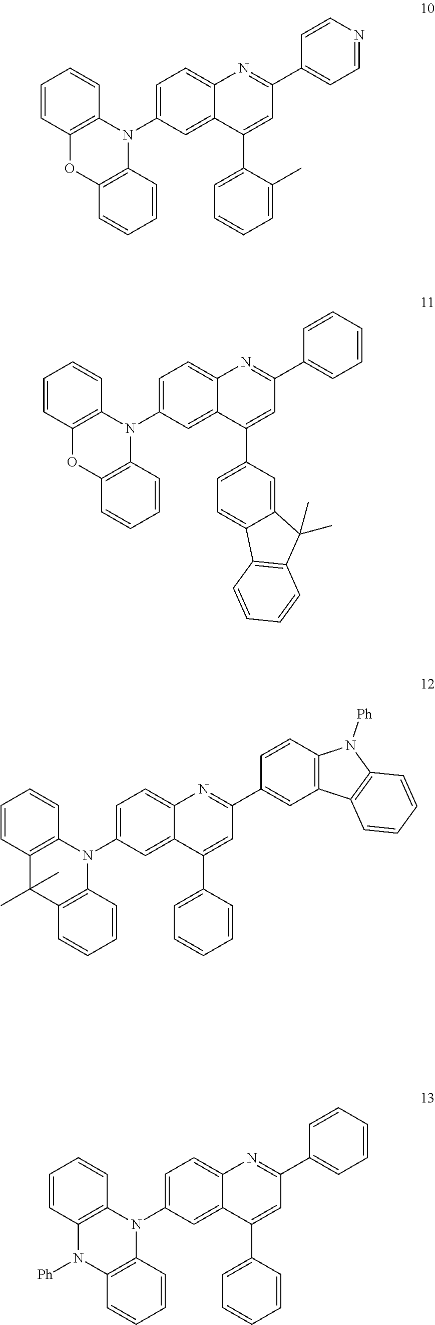

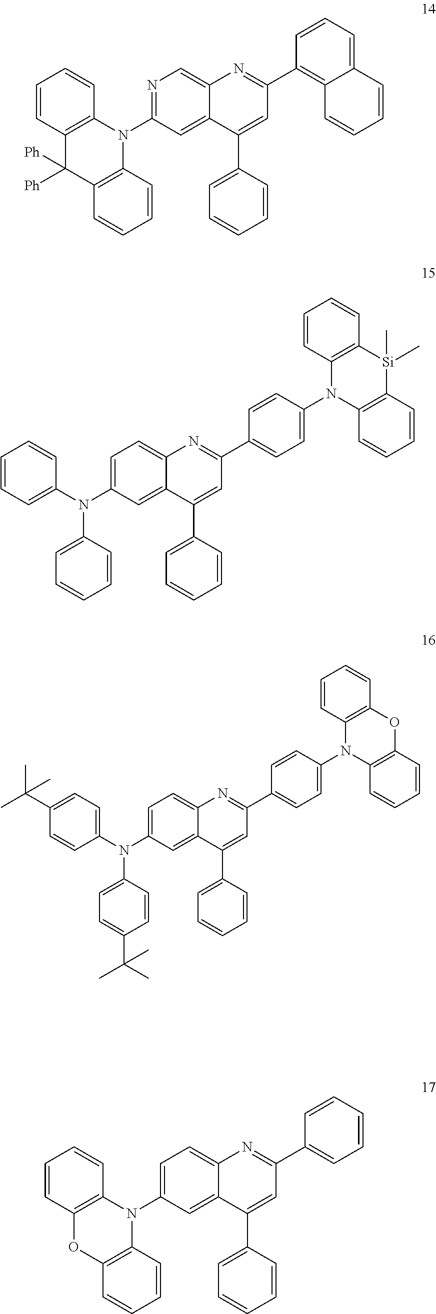

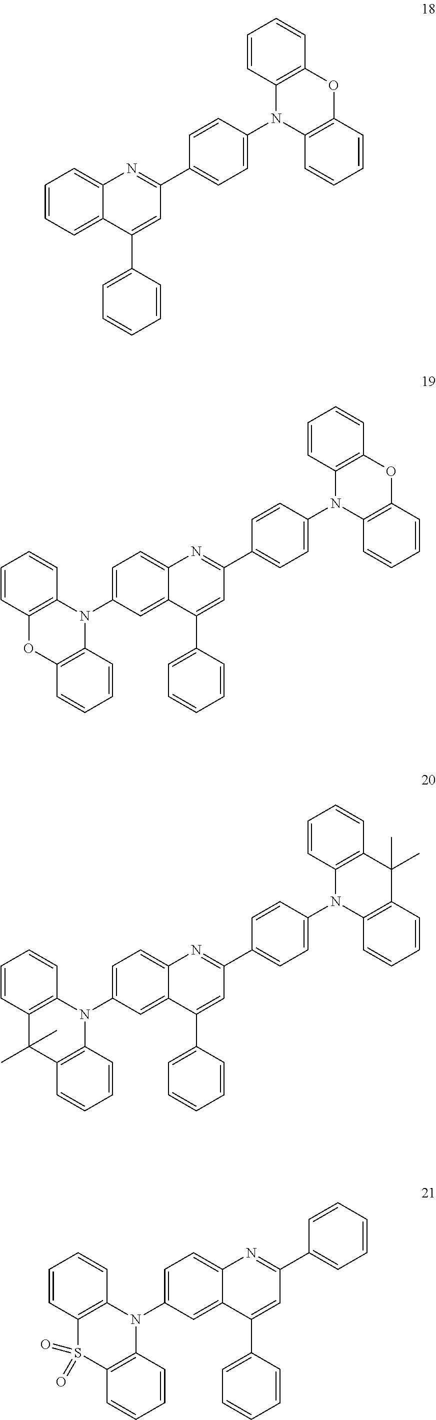

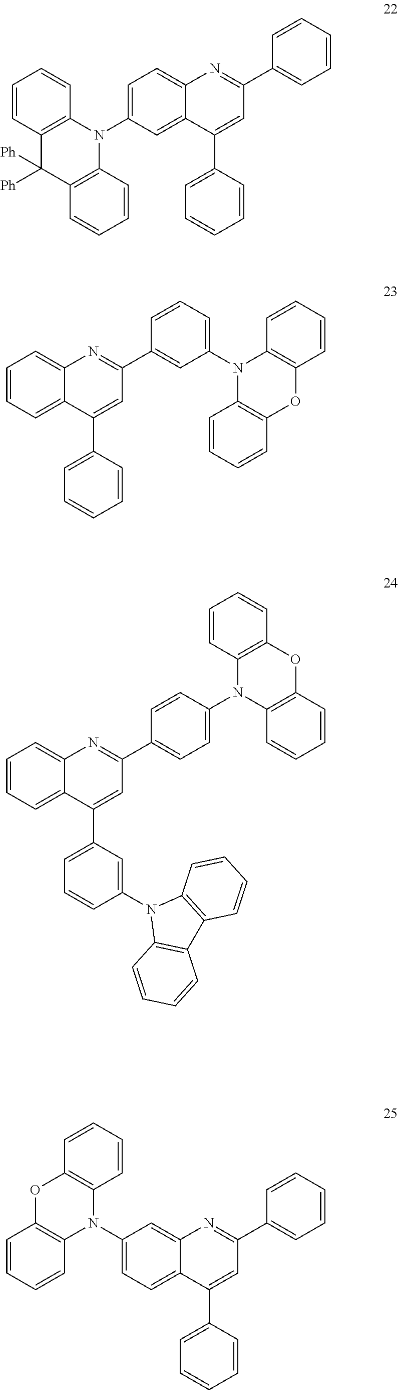 [SMILES: c1ccc(-c2cc(-c3ccccc3)c3cc(N4c5ccccc5C(c5ccccc5)(c5ccccc5)c5ccccc54)ccc3n2)cc1 ; c1ccc(-c2cc(-c3cccc(N4c5ccccc5Oc5ccccc54)c3)nc3ccccc23)cc1 ; c1cc(-c2cc(-c3ccc(N4c5ccccc5Oc5ccccc54)cc3)nc3ccccc23)cc(-n2c3ccccc3c3ccccc32)c1 ; c1ccc(-c2cc(-c3ccccc3)c3ccc(N4c5ccccc5Oc5ccccc54)cc3n2)cc1]

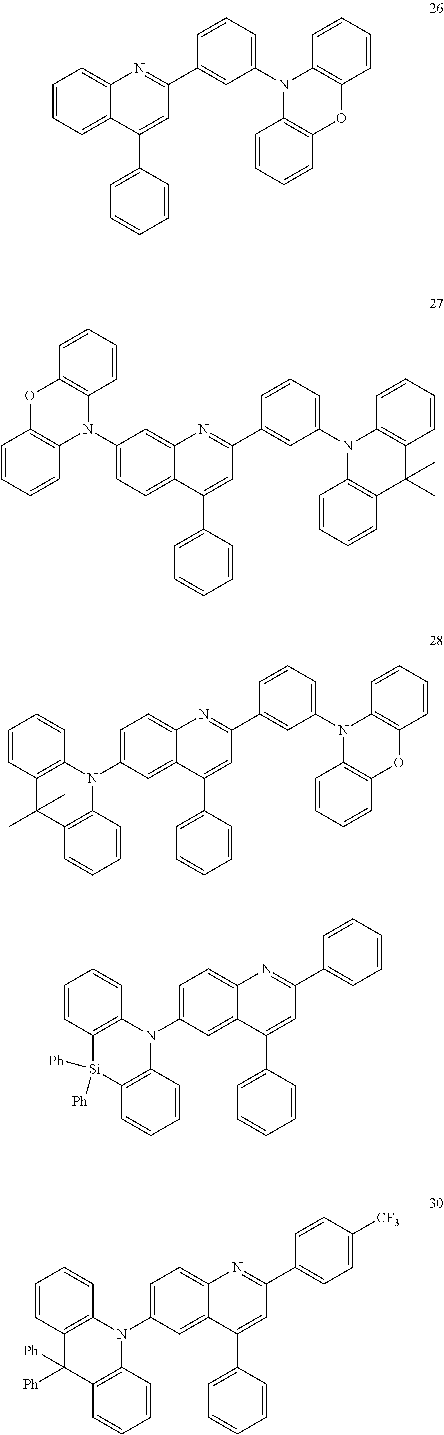

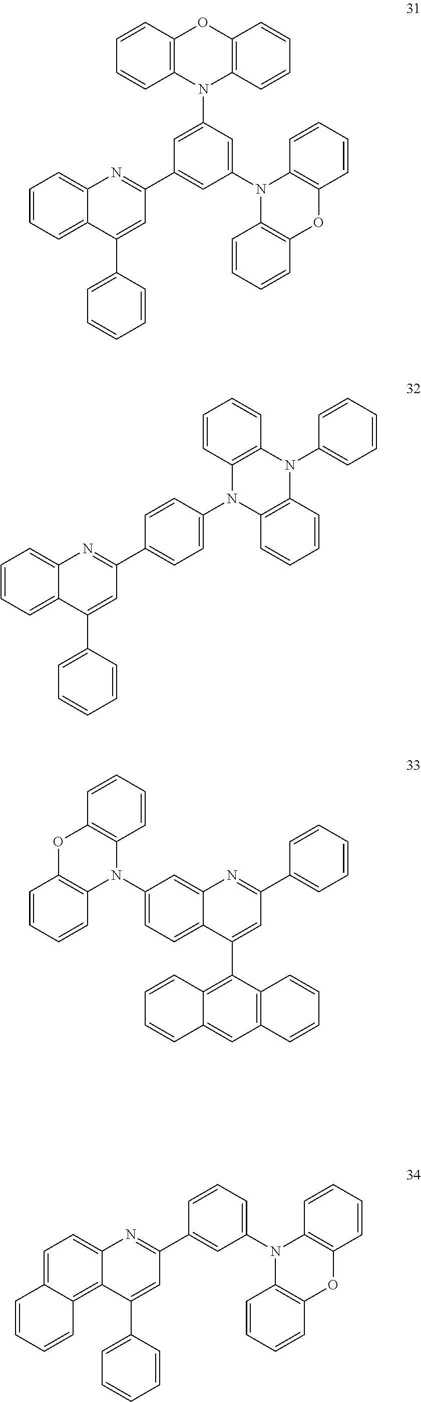 [SMILES: c1ccc(-c2cc(-c3cc(N4c5ccccc5Oc5ccccc54)cc(N4c5ccccc5Oc5ccccc54)c3)nc3ccccc23)cc1 ; c1ccc(-c2cc(-c3ccc(N4c5ccccc5N(c5ccccc5)c5ccccc54)cc3)nc3ccccc23)cc1 ; c1ccc(-c2cc(-c3c4ccccc4cc4ccccc34)c3ccc(N4c5ccccc5Oc5ccccc54)cc3n2)cc1 ; c1ccc(-c2cc(-c3cccc(N4c5ccccc5Oc5ccccc54)c3)nc3ccc4ccccc4c23)cc1]

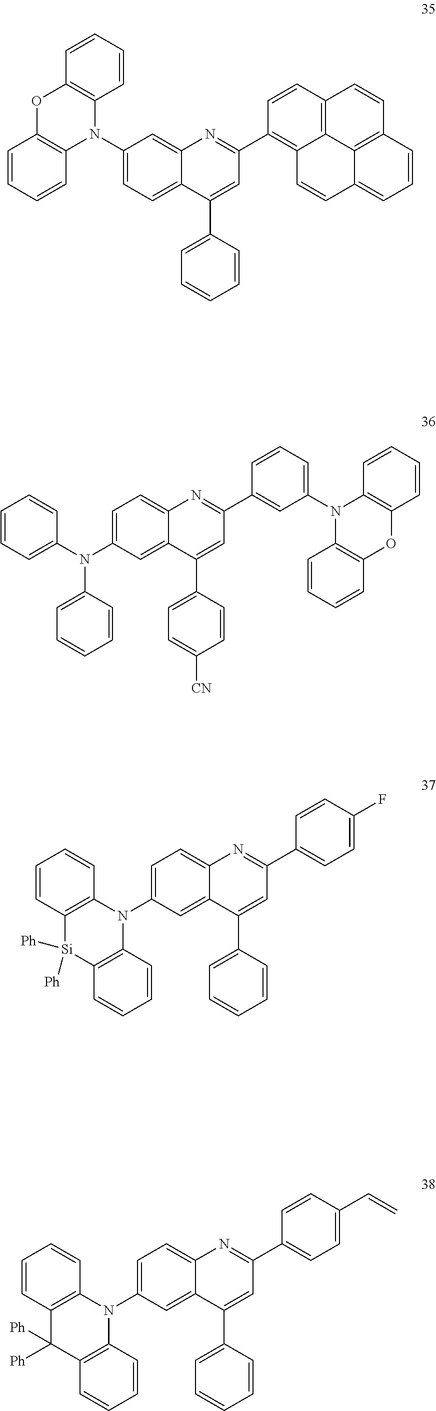 [SMILES: c1ccc(-c2cc(-c3ccc4ccc5cccc6ccc3c4c56)nc3cc(N4c5ccccc5Oc5ccccc54)ccc23)cc1 ; N#Cc1ccc(-c2cc(-c3cccc(N4c5ccccc5Oc5ccccc54)c3)nc3ccc(N(c4ccccc4)c4ccccc4)cc23)cc1 ; Fc1ccc(-c2cc(-c3ccccc3)c3cc(N4c5ccccc5[Si](c5ccccc5)(c5ccccc5)c5ccccc54)ccc3n2)cc1 ; C=Cc1ccc(-c2cc(-c3ccccc3)c3cc(N4c5ccccc5C(c5ccccc5)(c5ccccc5)c5ccccc54)ccc3n2)cc1]

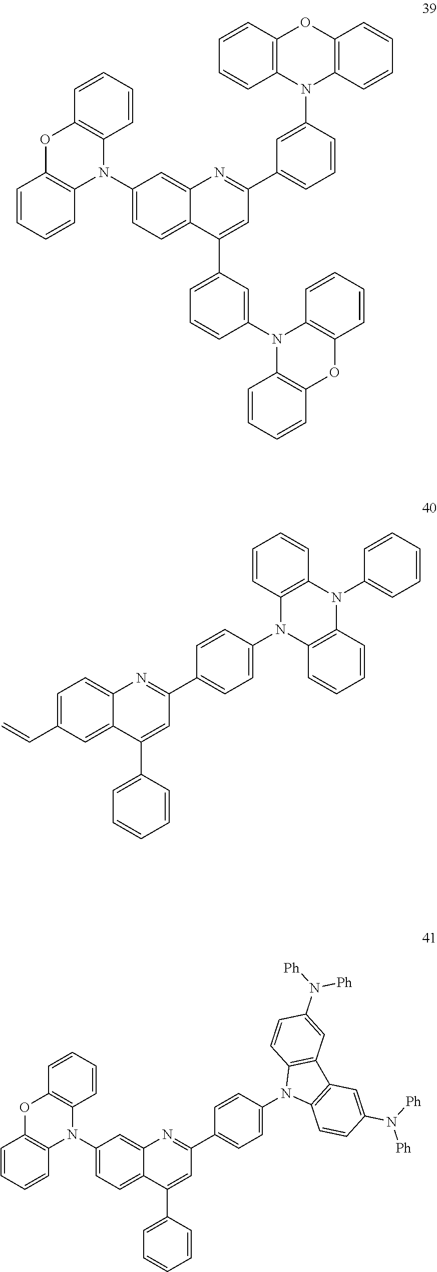 [SMILES: c1cc(-c2cc(-c3cccc(N4c5ccccc5Oc5ccccc54)c3)c3ccc(N4c5ccccc5Oc5ccccc54)cc3n2)cc(N2c3ccccc3Oc3ccccc32)c1 ; C=Cc1ccc2nc(-c3ccc(N4c5ccccc5N(c5ccccc5)c5ccccc54)cc3)cc(-c3ccccc3)c2c1 ; c1ccc(-c2cc(-c3ccc(-n4c5ccc(N(c6ccccc6)c6ccccc6)cc5c5cc(N(c6ccccc6)c6ccccc6)ccc54)cc3)nc3cc(N4c5ccccc5Oc5ccccc54)ccc23)cc1]

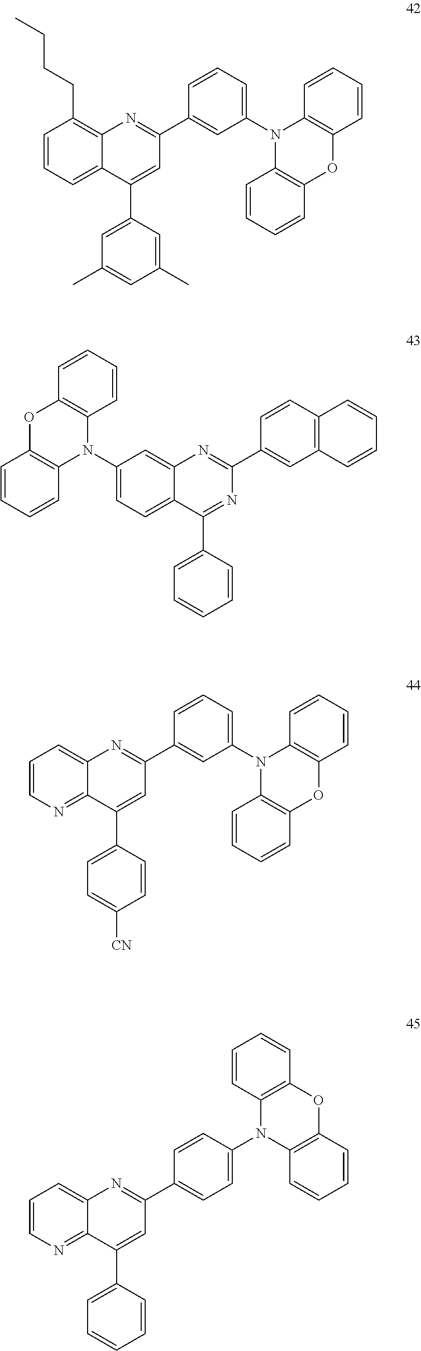 [SMILES: CCCCc1cccc2c(-c3cc(C)cc(C)c3)cc(-c3cccc(N4c5ccccc5Oc5ccccc54)c3)nc12 ; c1ccc(-c2nc(-c3ccc4ccccc4c3)nc3cc(N4c5ccccc5Oc5ccccc54)ccc23)cc1 ; N#Cc1ccc(-c2cc(-c3cccc(N4c5ccccc5Oc5ccccc54)c3)nc3cccnc23)cc1 ; c1ccc(-c2cc(-c3ccc(N4c5ccccc5Oc5ccccc54)cc3)nc3cccnc23)cc1]

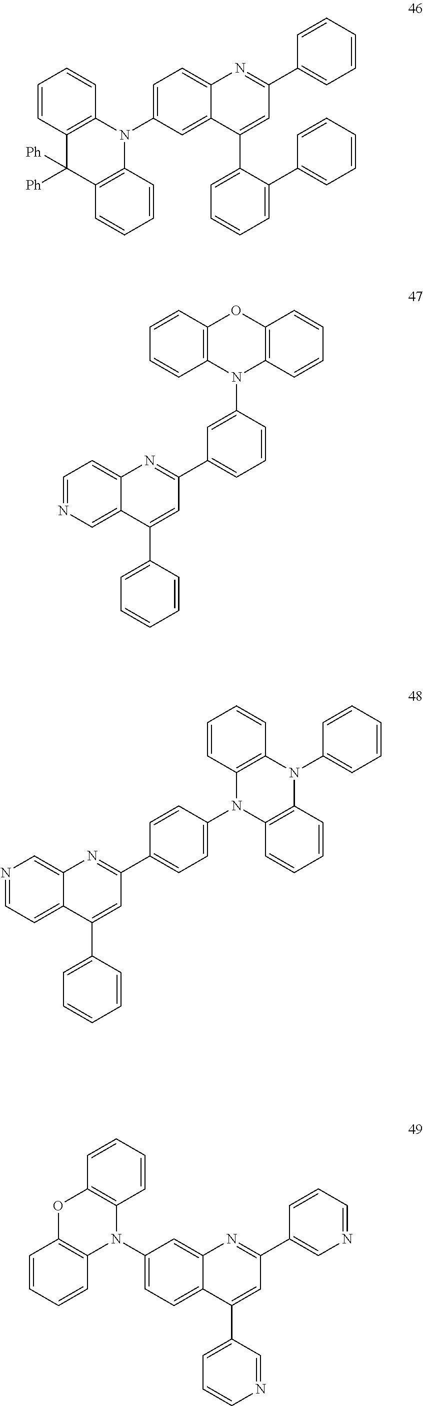 [SMILES: c1ccc(-c2cc(-c3ccccc3-c3ccccc3)c3cc(N4c5ccccc5C(c5ccccc5)(c5ccccc5)c5ccccc54)ccc3n2)cc1 ; c1ccc(-c2cc(-c3cccc(N4c5ccccc5Oc5ccccc54)c3)nc3ccncc23)cc1 ; c1ccc(-c2cc(-c3ccc(N4c5ccccc5N(c5ccccc5)c5ccccc54)cc3)nc3cnccc23)cc1 ; c1cncc(-c2cc(-c3cccnc3)c3ccc(N4c5ccccc5Oc5ccccc54)cc3n2)c1]

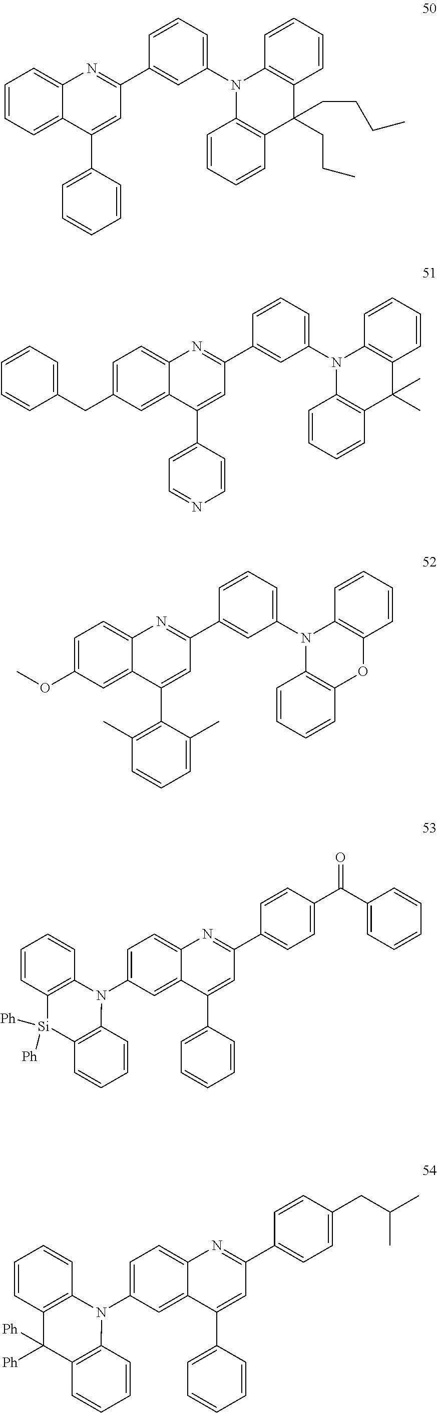

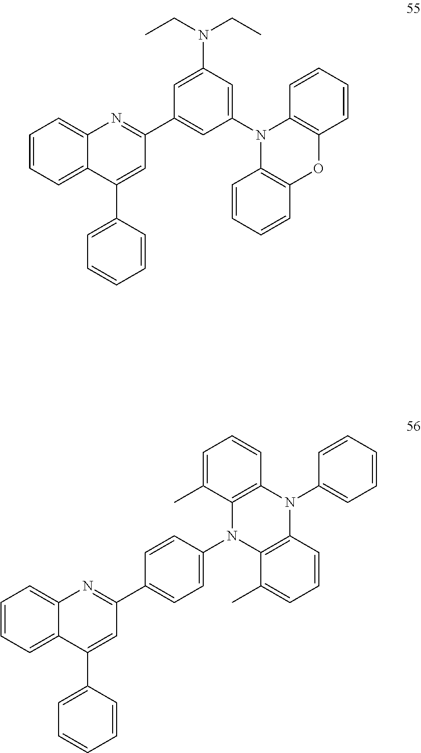 [SMILES: CCN(CC)c1cc(-c2cc(-c3ccccc3)c3ccccc3n2)cc(N2c3ccccc3Oc3ccccc32)c1 ; Cc1cccc2c1N(c1ccc(-c3cc(-c4ccccc4)c4ccccc4n3)cc1)c1c(C)cccc1N2c1ccccc1]

### Emission Materials & Electronic Devices

The nitrogen-containing aromatic compounds of Formula 1, as described herein including embodiments thereof as described herein, can be used as an active component in an electronic device.

The electronic device according to the present disclosure may comprise one or more organic layer(s) interposed between an anode and a cathode. The one or more organic layer(s) may each comprise or consist of one or more layers selected from an electron transport layer, an emission layer, a hole transporting layer, a hole injection layer, an insulation layers, and combination thereof. At least one of these layers includes a nitrogen-containing aromatic derivative of Formula 1 as described herein including any embodiments thereof.

The layer(s) may be constituted by (i) a single layer doped with a compound of formula 1; or (ii) multiple layers of which at least one layer may be doped with a compound of formula 1; or (iii) at least one layer is a layer comprised of a compound of formula 1 doped with a separate dopant; or (iv) multiple layers of which at least one layer may be comprised entirely of a compound of formula 1. The situation where a particular layer comprises primarily the compound of formula 1, and the layer is doped with at least one dopant may also be referred to as a host-guest material. In this situation, the compound of formula 1 is the guest material and the host is the host material.

Electronic devices may be selected from the group consisting of organic electroluminescent devices (OLEDs), organic field-effect transistors (O-FETs), organic thin-film transistors (O-TFTs), organic light-emitting transistors (O-LETs), organic integrated circuits (O-ICs), organic solar cells (O-SCs), organic field-quench devices (O-FQDs), light-emitting electrochemical cells (LECs), organic photoreceptors and organic laser diodes (O-lasers). In one embodiment, the electronic device is an organic electroluminescent device. The device may be a fluorescent OLED.

Active components are generally introduced between the anode and cathode, for example charge-injection, charge-transport or charge-blocking materials, but in particular emission materials and matrix materials. The nitrogen-containing aromatic compounds of Formula 1, as described herein including embodiments thereof as described herein, can exhibit particularly good properties for these functions, in particular as an TADF or a TTA emission material in organic electroluminescent devices, as described in further detail below. Organic electroluminescent devices are therefore a particular embodiment of the invention.

The organic electroluminescent device can comprise a cathode, an anode and at least one emitting layer (which may also be referred to as an emissive layer). The nitrogen-containing aromatic compounds of Formula 1, can be provided as an emitting compound in the emitting layer. The nitrogen-containing aromatic compounds of Formula 1, may also be provided as a host material for an emitting compound in the emitting layer.

The organic electronic device may comprise further layers, selected from in each case one or more hole-injection layers, hole-transport layers, hole-blocking layers, electron-transport layers, electron-injection layers, electron-blocking layers, exciton-blocking layers, charge-generation layers and/or organic or inorganic p/n junctions.

Optional interlayers may be added to provide, for example, an exciton-blocking function, to be introduced between two emitting layers or also between other layers. The fluorescent OLED may comprise one or more emitting layers, where at least one emitting layer comprises at least one nitrogen-containing aromatic compound of Formula 1. If a plurality of emission layers are present, these can have in total a plurality of emission maxima between 380 nm and 750 nm, resulting overall in white emission, i.e. various emitting compounds which are able to fluoresce or phosphoresce are used in the emitting layers. There may be provided a three-layer system, where the three layers exhibit blue, green and orange or red emission, for example in accordance with WO05/011013.

In one embodiment, the electronic device comprises at least one nitrogen-containing aromatic compound of Formula 1, as an emitting compound in an emitting layer. Each of the one or more layers in the device may comprise one or more matrix materials. The mixture of the nitrogen-containing aromatic compounds of the Formula 1, and the matrix material may comprise between 1 and 99% by vol., between 2 and 90% by vol., between 3 and 40% by vol., or between 5 and 15% by vol., of the metal complex, based on the mixture as a whole comprising emitter and matrix material. Correspondingly, the mixture may comprise between 99 and 1% by vol., between 98 and 10% by vol., between 97 and 60% by vol., or between 95 and 85% by vol., of the matrix material, based on the mixture as a whole comprising emitter and matrix material.

Suitable host materials may include ketones, phosphine oxides, sulfoxides and sulfones, for example in accordance with WO04/013080, WO04/093207 or WO06/005627, triarylamines, carbazole derivatives, for example CBP (N,N-biscarbazolylbiphenyl), mCBP or the carbazole derivatives disclosed in WO05/039246, US2005/0069729, JP2004/288381, EP1205527 or WO08/086851, poly-CBP or poly-CBP-fluorene disclosed in WO09/080799, polyfluorene derivatives or polyvinylarylene derivatives, for example poly(p-phenylenevinylene) disclosed in JP2004288632, indolocarbazole derivatives, for example in accordance with WO07/063754 or WO08/056746, azacarbazoles, for example in accordance with EP1617710, EP1617711, EP1731584, JP2005/347160, bipolar host materials, for example in accordance with WO07/137725, silanes, for example in accordance with WO 05/111172, azaboroles or boronic esters, for example in accordance with WO06/117052, triazine derivatives, for example in accordance with WO07/063754 or WO08/056746, or zinc complexes, for example in accordance with EP652273 or WO09/062578. The nitrogen-containing aromatic compounds of Formula 1, may also be suitable as host materials. In general, all host materials as employed in accordance with the prior art for fluorescent or phosphorescent emitters in organic electroluminescent devices can also be employed for the compounds according to the invention. Mixtures of these matrix materials may also be used.

The electroluminescent device may comprise a separate hole-transport layer or comprise a nitrogen-containing aromatic compound, which is identical or similar to the compound employed in the emitting layer, as hole-transport material in the hole-transport layer.

The nitrogen-containing aromatic compound of Formula 1, including embodiments as described herein, may be employed as a host material for an emitting compound in an emitting layer.

The electroluminescent device may comprise a host material and at least two fluorescent emitters in the emitting layer, where at least one of the two fluorescent emitters is a nitrogen-containing aromatic compound of Formula 1. The fluorescent emitter which emits at shorter wavelength serves here as host for the fluorescent emitter which emits at longer wavelength. The nitrogen-containing aromatic compound of Formula 1, may be the compound emitting at shorter wavelength or the compound emitting at longer wavelength. Likewise, both fluorescent compounds can be a nitrogen-containing aromatic compound of Formula 1.

The nitrogen-containing aromatic compounds of Formula 1, as described herein, may be used as hole-blocking materials in a hole-blocking layer and/or as electron-transport material in an electron-transport layer. The emitting layer may be fluorescent or phosphorescent.

Each of the one or more layers in the organic electroluminescent device may be prepared by a sublimation process or by organic vapour phase deposition process, such as organic vapour jet printing (see M. S. Arnold et al., Appl. Phys. Lett. 2008, 92, 053301). Each of the one or more layers in the organic electroluminescent device may be produced from solution, such as, for example, by spin coating, or by means of any desired printing process, such as, for example, screen printing, flexographic printing, offset printing, LITI (light induced thermal imaging, thermal transfer printing), ink-jet printing or nozzle printing. Hybrid processes, in which one or more layers are applied from solution and one or more other layers are applied by vapour deposition, are also possible. Each of the one or more layers in the organic electroluminescent device may be prepared by a solution process or an evaporation process in vacuo, or in combination.

In some embodiments, the nitrogen-containing aromatic compounds of Formula 1, as described herein, may be used in combination with one or more of a hole injection material, a hole transporting compound (or material), an electron transporting compound and/or an additional emission compound, examples of which may include the following:

Exemplary hole transporting materials/compounds include:

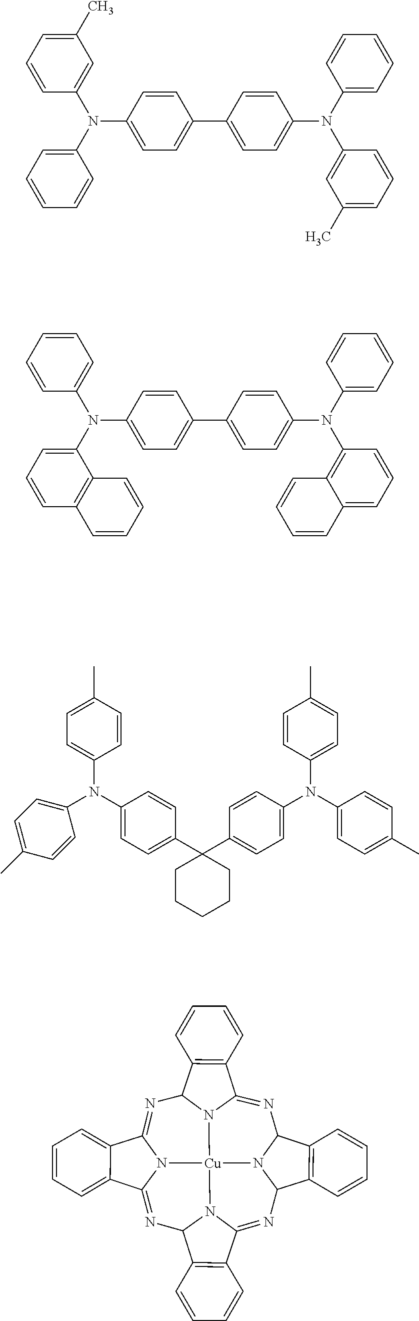

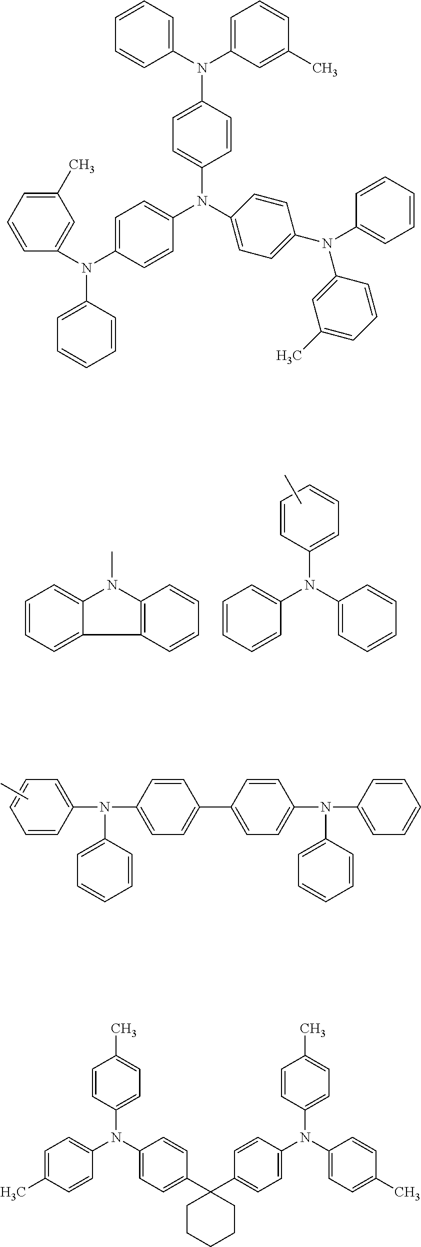

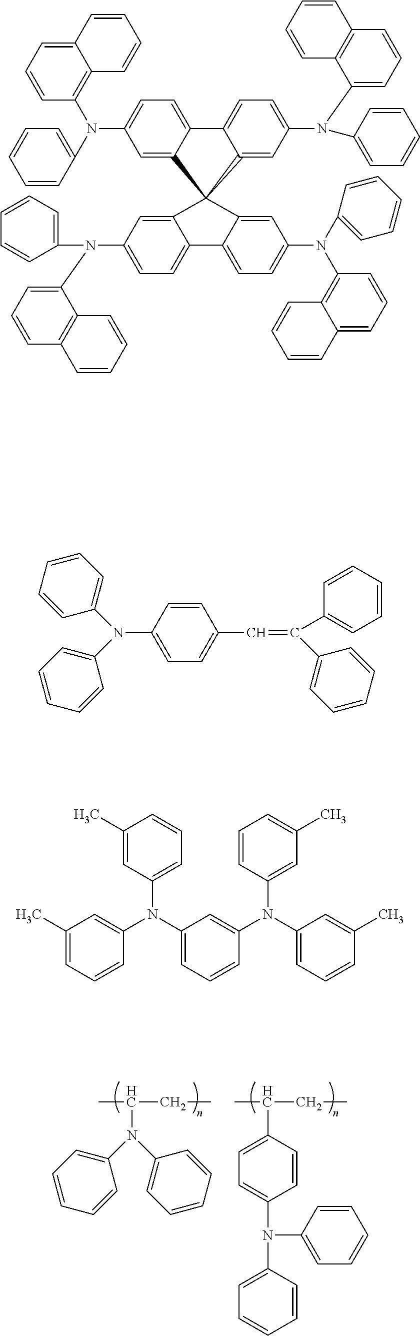

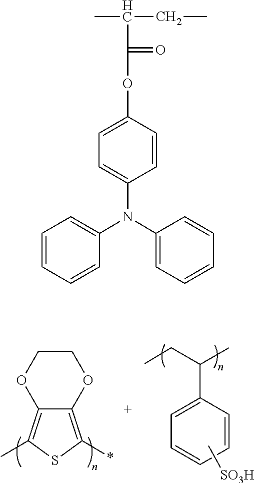

Exemplary electron transporting materials/compounds include:

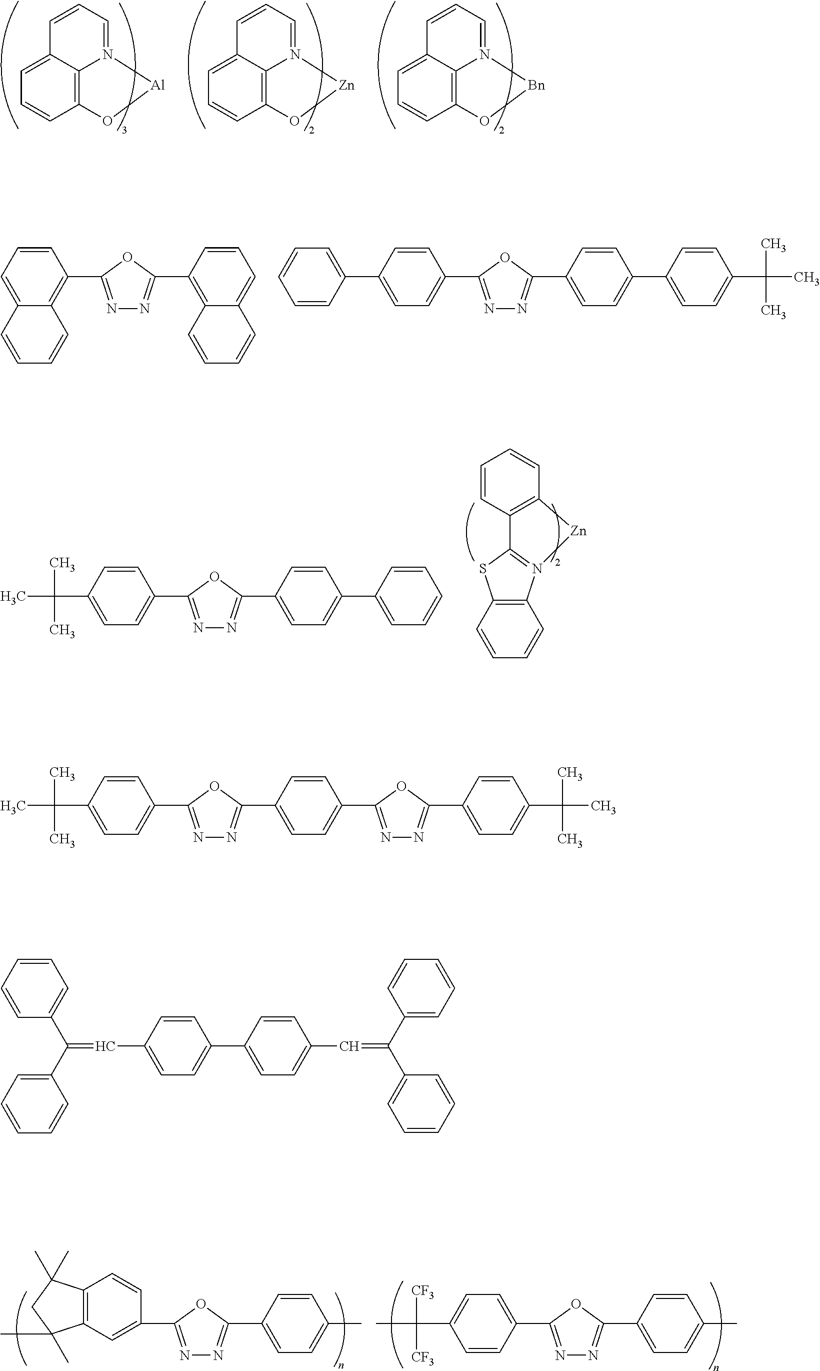

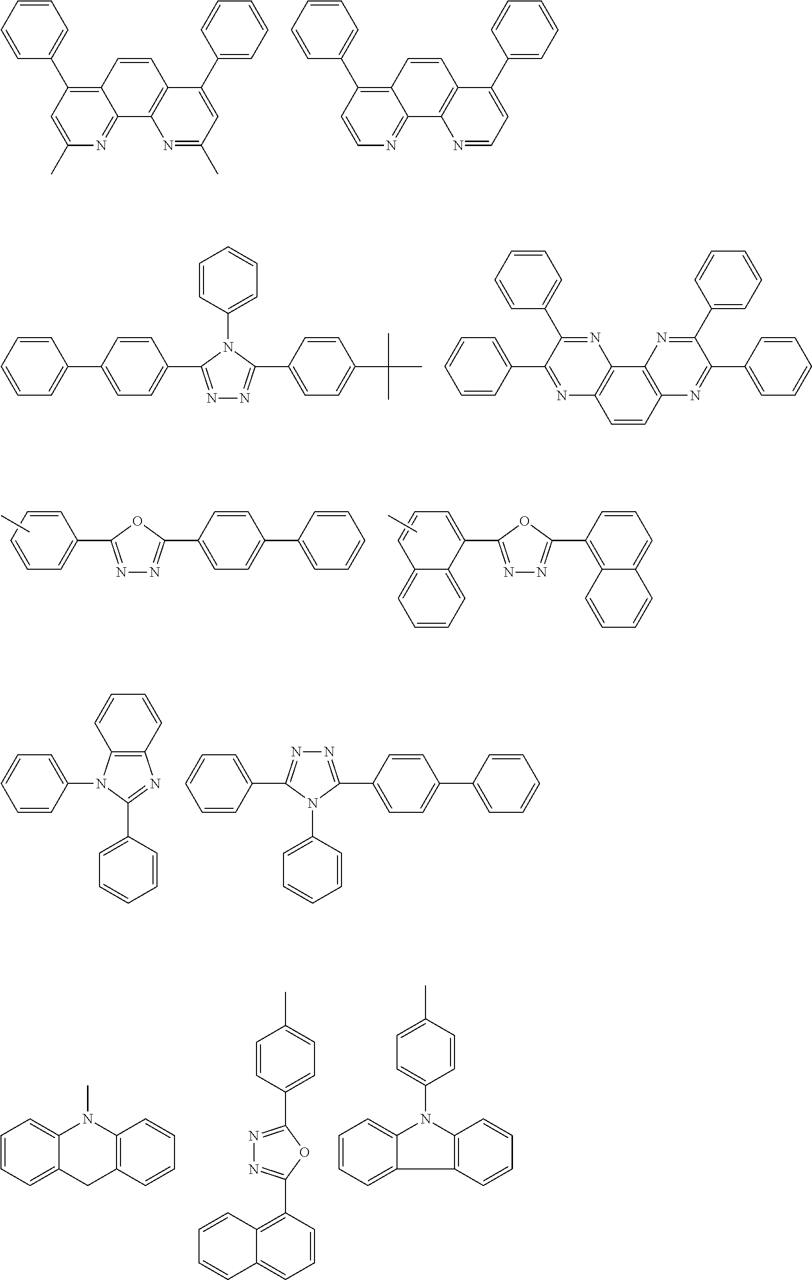

The anode is typically selected from a material having a large work function, examples of which may include metals, such as gold, platinum, nickel, palladium, cobalt, selenium, vanadium and their alloys; metal oxides, such as tin oxide, zinc oxide, indium zinc oxide (IZO) and indium tin oxide (ITO) and electroconductive polymers, such as PEDOT:PSS, polyaniline, polypyrrole and polythiophene and derivatives thereof. These compounds may be used singly or in combination of two or more species. A substrate for the anode of the organic electroluminescence device may be provided and selected from an opaque substrate made from any suitable material, such as metal or ceramics, or a transparent substrate made from any suitable transparent material such as glass, quartz, plastics.

The cathode is typically selected from a material having a smaller work function, usually under 4.0 eV, examples of which may include; metals such as sodium, magnesium, calcium, lithium, potassium, aluminium, indium, silver, lead, chromium and their alloys, or oxides.

A charge blocking layer may be deposited adjacent to either electrode to avoid current leakage. The charge blocking material may be an inorganic compound, examples of which may include aluminium oxide, lithium fluoride, lithium oxide, caesium oxide, magnesium oxide, magnesium fluoride, calcium oxide, calcium fluoride, aluminium nitride, titanium oxide, silicon oxide, silicon nitride, boron nitride, vanadium oxide.

The one or more organic layers in the electronic device may be constituted by:

a single layer doped with a fluorescent material of the present application;

multiple layers of which at least one layer may be doped with a fluorescent material of the present application; or

multiple layers of which at least one layer may be comprised entirely of a fluorescent material of the present application.

Further examples of the device structure may be provided as follows:

anode/emissive layer/cathode;

anode/electron transport layer/cathode;

anode/hole transporting layer/emissive layer/electron transporting layer/cathode;

anode/hole injection layer/hole transporting layer/emissive layer/electron transporting layer/cathode;

anode/hole transporting layer/emission layer/electron transporting layer/electron injection layer/cathode;

anode/hole injection layer/emission layer/electron transporting layer/electron injection layer/cathode;

anode/charge blocking layer/hole transporting layer/emission layer/electron transporting layer/cathode;

anode/hole transporting layer/emission layer/electron transporting layer/charge blocking layer/cathode;

anode/inorganic semiconductor/ charge blocking layer/hole transporting layer/emission layer/charge blocking layer/cathode;

anode/charge blocking layer/hole transporting layer/emission layer/electron transporting layer/charge blocking layer/cathode;

anode/charge blocking layer/hole injection layer/hole transporting layer/emission layer/electron transporting layer/electron injection layer/cathode; or

anode/charge blocking layer/hole injection layer/hole transporting layer/emission layer/electron transporting layer/electron injection layer/charge blocking layer/cathode.

### Polymers and Polymerizable Agents

There may be provided a polymer comprising a compound of Formula 1 or any embodiment thereof as described above or herein. The compounds of Formula 1 may be incorporated into the polymer. The compounds of Formula 1 may be covalently bonded to the polymer. The compounds of Formula 1 may be used as polymerizable monomers or as pendant groups in polymerizable monomers. The compounds of Formula 1 may be used as prepolymers, for example in prepolymer compositions. The polymer or polymer material may be used with other materials. The polymer or polymer material may be provided in one or more layers of an electroluminescent device as described herein.

### Imaging and Sensitizer Agents

There may be provided an imaging agent selected from a compound of Formula 1 or any embodiment thereof as described above or herein. There may be provided a pharmaceutical composition comprising the imaging agent. The imaging agents may comprise any pharmaceutically acceptable salts of the compounds of Formula 1. The imaging agents may be used in diagnostic methods.

There may be provided a sensitizer material comprising a compound of Formula 1 or any embodiment thereof as described above or herein. The sensitizer material may be for therapeutic or diagnostic use. There may be provided a pharmaceutical composition comprising the sensitizer material. The sensitizer material may comprise any pharmaceutically acceptable salts of the compounds of Formula 1. In an eighth aspect, there is provided a polymer comprising a compound of Formula 1 or any embodiment thereof as described above or herein. The compounds of Formula 1 may be incorporated into the polymer. The compounds of Formula 1 may be covalently bonded to the polymer. The compounds of Formula 1 may be used as polymerizable monomers or as pendant groups in polymerizable monomers.

It will be appreciated that in other aspects there may be provided a use, method or process comprising a compound of Formula 1 or any embodiment thereof, as described above or herein.

### Photo-Dynamic Therapy (PDT)

Conventional photo-dynamic therapy (PDT) is a two-step process involving the application of a photosensitive agent followed by controlled exposure to a selective light source which activates the photosensitive agent. The photosensitive agent can be applied internally or externally to an area of the body which needs to be treated. PDT may be used in treatment of skin cancer including; basal cell carcinoma (BCC), actinic (solar) keratosis (AK) and bowens disease (squamous carcinoma in situ). PDT may be used in dermatology including nonmelanoma skin cancer, acne vulgaris, photorejuvenation, and hidradenitis suppurativa. PDT may also be used in the treatment of psoriasis, lichen planus, lichen sclerosus, scleroderma, cutaneous T cell lymphoma, alopecia areata, verruca vulgaris, darier's disease and tinea infections.

The photosensitive agent will only work after it has been activated by a light source. Depending on the part of the body being treated, the photosensitizing agent is either put into the bloodstream through a vein or put on the skin. Over a certain amount of time the photosensitising agent is absorbed. This amount of time can be anywhere from about three hours to a couple of days, depending on the photosensitising agent used. Then light is applied to the area to be treated. The light causes the photosensitising agent to react with oxygen to form highly reactive intermediaries. These have a short half-life (fractions of a second) and thus have a very localised tissue-damaging effect. Several photosensitizing agents are currently available. For example, porfimer sodium (Photofrin®), aminolevulinic acid (ALA or Levulan®) and methyl ester of ALA (Metvixia® cream).

The conventional procedure required for PDT involve the patient going to the hospital to be administered the photosensitive agent, for example cream or injection. Once administered the patient is required to wait several hours to a couple of days before a light of suitable wavelength is supplied to the photosensitive agent. Lasers or filtered arc lamps are used as the light source, which are unwieldy, inconvenient, expensive to the patient, and this technique is restricted to hospital use.

In an embodiment, the electroluminescent device is as described herein comprises a light source for use in a therapeutic and/or cosmetic treatment, which in use, covers the area to be treated and emits light to activate the photosensitive agent and initiate treatment. In an embodiment, the electroluminescent device is sufficiently portable so that, in use, the patient can move around freely. This provides the patient with convenience and avoidance of hospital stays. This means that a lower light level can be used since exposure time can be increased. As a result of the lower light level, the pain associated with the treatment (i.e. caused by high irradiances) is reduced. The electroluminescent device is lightweight and can be powered by a portable low voltage power supply. At least according to some embodiments, the compounds as described herein may provide further advantage in relation to suitable processing to form effective thin film portable devices.

In an embodiment, the electroluminescent device is for use in the treatment of a human or animal by photodynamic therapy or cosmetic applications. In an embodiment, the device comprises an external quantum efficiency of more than about 20%, more than about 19%, more than about 18%, more than about 17%, more than about 16%, more than about 15%, more than about 14%, more than about 13%, more than about 12%, more than about 11%, more than about 10%, more than about 9.0%, more than about 8.0%, more than about 7.0%, more than about 6.0%, more than about 5.0%, more than about 4.0%, more than about 3.0%. In a particular embodiment, the device comprises an external quantum efficiency of more than about 5%.

In an embodiment, the electroluminescent device may comprise one or more transparent layers. For example, one or more of the cathode, anode, and emitting layer, hole-injection layer, hole-transport layer, hole-blocking layer, electron-transport layers, electron-injection layers, electron-blocking layers, exciton-blocking layers, charge-generation layers and/or organic or inorganic p/n junctions may be transparent. The transparency of the one or more layers may be greater than about 60%, greater than about 65%, greater than about 70%, greater than about 75%, greater than about 80%, greater than about 85%, greater than about 90%, and greater than about 99%. In one particular embodiment, the transparency is greater than 80%. The transparency of the one or more layers may be between about 60-99%, between about 65-95%, between about 70-90%, or between about 75-85%.

In an embodiment, the electroluminescent device is flexible. For example, the device may be formed into a variety of different configurations so as to be capable of shaping to the body part for which it is applied. In a specific example, the electroluminescent device may be attached to a stretching bandage (e.g. VELCRO) or installed inside a facemask for face cancer therapy.

### Synthesis of Nitrogen-Containing Aromatic Compounds

The compounds of Formula 1 can be prepared by various processes, but where the processes described below have proven particularly suitable.

### EXAMPLES

### Example 1

### Synthesis of Compound of Nitrogen-Containing Aromatic Compound of Formula 1

### Synthesis of 10-(2,4-diphenylquinolin-6-yl)-10H-phenoxazine

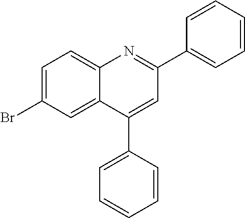 [SMILES: Brc1ccc2nc(-c3ccccc3)cc(-c3ccccc3)c2c1]

### 6-bromo-2,4-diphenylquinoline

Following the general, previously described procedure (Chem. Eur. J. 2009, 15, 6332-6334), a mixture of phenylacetylene (1.65 mL, 15 mmol), 4-bromoaniline (1.81 g, 10.5 mmol), benzaldeyde (1.02 mL, 10.0 mmol) and FeCl₃ (160 mg, 1.0 mmol) in toluene (10 mL) was heated to 100° C. for 24 h. The mixture was allowed to cool to room temperature and then filtered through silica gel washing with EtOAc/CH₂Cl₂/petrol (5:20:75). The filtrate was concentrated to give a solid residue. The residue was purified by silica gel column chromatography (EtOAc/petrol 3:97 then 4:96) to give 6-bromo-2,4-diphenylquinoline (1.75 g, 48%) as colourless solid. A portion of this material was further purified by recrystallisation (toluene/petrol) to give colourless crystals. ¹H NMR (CDCl₃, 400 MHz) δ 7.45-7.61 (m, 8H), 7.80 (dd, J 2.2, 9.0 Hz, 1H), 7.84 (s, 1H), 8.04 (d, J 2.2 Hz, 1H), 8.11 (d, J 9.0 Hz, 1H), 8.16-8.21 (m, 2H); ¹³C NMR (CDCl₃, 100 MHz) δ 120.03, 120.43, 126.98, 127.54, 127.78, 128.72, 128.82, 128.90, 129.43, 129.63, 131.81, 133.02, 137.68, 139.14, 147.38, 148.40, 157.18.

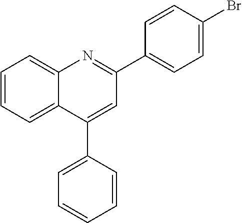 [SMILES: Brc1ccc(-c2cc(-c3ccccc3)c3ccccc3n2)cc1]

### 2-(4-bromophenyl)-4-phenylquinoline

A mixture of phenylacetylene (2.47 mL, 22.5 mmol), aniline (1.44 mL, 15.8 mmol), 4-bromobenzaldeyde (2.78 g, 15.0 mmol) and FeCl₃ (240 mg, 1.50 mmol) in toluene (15 mL) was heated to 100° C. for 24 h. The mixture was allowed to cool to room temperature and then filtered through silica gel washing with EtOAc/CH₂Cl₂/petrol (5:20:75). The filtrate was concentrated to give a solid residue. The residue was purified by silica gel column chromatography (EtOAc/petrol 3:97 then 4:96) to give 2-(4-bromophenyl)-4-phenylquinoline (1.50 g, 28%) as colourless solid. A portion of this material was further purified by recrystallisation (toluene/petrol) to give colourless crystals. ¹H NMR (CDCl₃, 400 MHz) δ 7.47-7.58 (m, 6H), 7.64-7.68 (m, 2H), 7.75 (ddd, J 1.4, 6.8 8.4 Hz, 1H), 7.78 (s, 1H), 7.91 (dd, J 0.9, 8.4 Hz, 1H), 8.07-8.12 (m, 2H), 8.24 (dd, J0.5, 8.5 Hz, 1H); ¹³C NMR (CDCl₃, 100 MHz) δ 118.84, 123.96, 125.68, 125.83, 126.57, 128.50, 128.62, 129.10, 129.52, 129.72, 130.04, 131.95, 138.21, 138.42, 148.71, 149.46, 155.54.

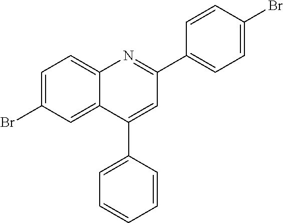 [SMILES: Brc1ccc(-c2cc(-c3ccccc3)c3cc(Br)ccc3n2)cc1]

### 6-bromo-2-(4-bromophenyl)-4-phenylquinoline

A mixture of phenylacetylene (2.47 mL, 22.5 mmol), 4-bromoaniline (2.71 g, 15.8 mmol), 4-bromobenzaldeyde (2.78 g, 15.0 mmol) and FeCl₃ (240 mg, 1.50 mmol) in toluene (15 mL) was heated to 100° C. for 24 h. The mixture was allowed to cool to room temperature and then filtered through silica gel washing with EtOAc/CH₂Cl₂/petrol (8:30:62). The filtrate was concentrated to give a solid residue. The residue was purified recrystallisation (toluene/petrol) to give 6-bromo-2-(4-bromophenyl)-4-phenylquinoline (3.5 g, 53%) as colourless crystals. ¹H NMR (CDCl₃, 400 MHz) δ 7.50-7.61 (m, 5H), 7.63-7.67 (m, 2H), 7.79 (s, 1H), 7.80 (dd, J 2.2, 9.0 Hz, 1H), 8.03 (d, J 2.1 Hz, 1H), 8.05-8.10 (m, 3H); ¹³C NMR (CDCl₃, 100 MHz) δ 119.50, 120.70, 124.27, 127.03, 127.81, 128.81, 128.86, 129.03, 129.39, 131.76, 132.02, 133.20, 137.51, 137.96, 147.33, 148.61, 155.83.

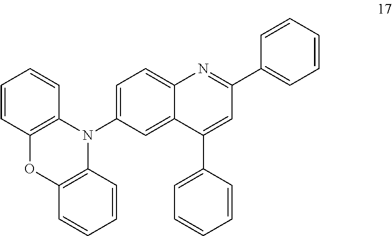 [SMILES: c1ccc(-c2cc(-c3ccccc3)c3cc(N4c5ccccc5Oc5ccccc54)ccc3n2)cc1]

### 10-(2,4-diphenylquinolin-6-yl)-10H-phenoxazine 17

A mixture of 6-bromo-2,4-diphenylquinoline (910 mg, 2.53 mmol), 10H-phenoxazine (532 mg, 2.91 mmol) and NaO^{t}Bu (340 mg, 3.54 mmol) in toluene (30 mL) was degassed (N₂ bubbling, 15 min). Tris(dibenzylideneacetone)dipalladium(0) (20 mg, 0.022 mmol) and 1,1′-bis(diphenylphosphino)ferrocene (24 mg, 0.044 mmol) were added and the mixture was heated to reflux (24 h). The mixture was allowed to cool to room temperature and then filtered through silica gel washing with EtOAc/CH₂Cl₂/petrol (8:30:62). The filtrate was concentrated to give a solid residue. The residue was purified by silica gel column chromatography (EtOAc/petrol 3:97 then 4:96) to give 10-(2,4-diphenylquinolin-6-yl)-10H-phenoxazine 17 (1.01 g, 86%) as colourless solid. A portion of this material was further purified by, firstly, recrystallisation (CH₂Cl₂/petrol) and, secondly, by (tube furnace) distillation (210° C., 10⁻⁶ mBar): m.p. 173-178° C. (DSC). ¹H NMR (CDCl₃, 400 MHz) δ 5.96 (dd, J 1.5, 7.9 Hz, 2H), 6.60 (dt, J 1.5, 6.8 Hz, 2H), 6.66 (dt, J 1.5, 7.8 Hz, 2H), 6.71 (dd, J 1.6, 7.8 Hz, 2H), 7.47-7.60 (m, 8H), 7.68 (dd, J 2.3, 8.9 Hz, 1H), 7.90 (s, 1H), 7.95 (d, J 2.2 Hz, 1H), 8.22-8.26 (m, 2H), 8.49 (d, J 8.8 Hz, 1H); ¹³C NMR (CDCl₃, 100 MHz) δ 113.22, 115.52, 119.84, 121.49, 123.26, 127.16, 127.62, 127.98, 128.75, 128.89, 128.94, 129.44, 129.68, 131.82, 133.71, 134.23, 136.44, 137.65, 139.27, 143.93, 148.33, 149.24, 157.85; HRMS (APCI) m/z 463.1801 C₃₃H₂₃ON₂ [M+H]⁺⁺ requires 463.1805.

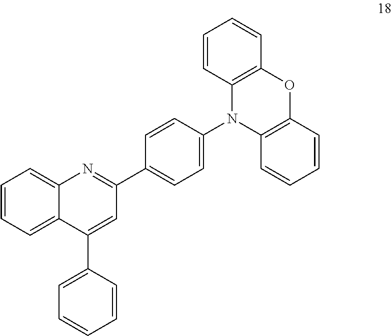 [SMILES: c1ccc(-c2cc(-c3ccc(N4c5ccccc5Oc5ccccc54)cc3)nc3ccccc23)cc1]

### 10-(4-(4-phenylquinolin-2-yl)phenyl)-10H-phenoxazine 18

A mixture of 2-(4-bromophenyl)-4-phenylquinoline (830 mg, 2.31 mmol), 10H-phenoxazine (485 mg, 2.65 mmol) and NaO^{t}Bu (310 mg, 3.23 mmol) in toluene (30 mL) was degassed (N₂ bubbling, 15 min). Tris(dibenzylideneacetone)dipalladium(0) (21 mg, 0.023 mmol) and 1,1′-bis(diphenylphosphino)ferrocene (26 mg, 0.046 mmol) were added and the mixture was heated to reflux (24 h). The mixture was allowed to cool to room temperature and then filtered through silica gel washing with EtOAc/CH₂Cl₂/petrol (8:30:62). The filtrate was concentrated to give a solid residue. The residue was purified by silica gel column chromatography (EtOAc/petrol 2:98 then 3:97) to give 10-(4-(4-phenylquinolin-2-yl)phenyl)-10H-phenoxazine 18 (850 mg, 80%) as colourless solid. A portion of this material was further purified by, firstly, recrystallisation (CH₂Cl₂/petrol) and, secondly, by (tube furnace) sublimation (220° C., 10⁻⁶ mBar): m.p. 222-128° C. (DSC). ¹H NMR (CDCl₃, 400 MHz) δ 6.05 (d, J 7.8 Hz, 2H), 6.57-6.74 (m, 6H), 7.49-7.63 (m, 8H), 7.78 (t, J 7.3 Hz, 1H), 7.88 (s, 1H), 7.96 (d, J 8.4 Hz, 1H), 8.29 (d, J 8.4 Hz, 1H), 8.42 (d, J 8.3 Hz, 2H); ¹³C NMR (CDCl₃, 100 MHz) δ 113.36, 115.46, 119.31, 121.40, 123.25, 125.72, 125.88, 126.66, 128.54, 128.65, 129.54, 129.76, 130.15, 130.30, 131.26, 134.21, 138.22, 139.89, 140.00, 143.95, 148.83, 149.55, 155.90; HRMS (APCI) m/z 462.1728 C₃₃H₂₂ON₂ [M]^{+·} requires 462.1727.

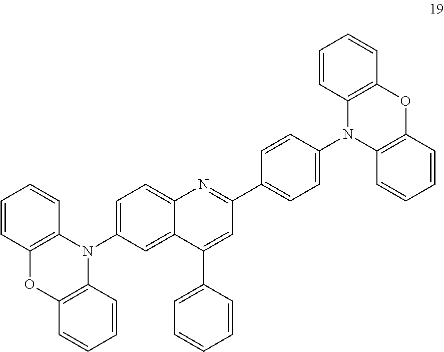 [SMILES: c1ccc(-c2cc(-c3ccc(N4c5ccccc5Oc5ccccc54)cc3)nc3ccc(N4c5ccccc5Oc5ccccc54)cc23)cc1]

### 10-(4-(6-(10H-phenoxazin-10-yl)-4-phenylquinolin-2-yl)phenyl)-10H-phenoxazine 19

A mixture of 6-bromo-2-(4-bromophenyl)-4-phenylquinoline (1.40 g, 3.21 mmol), 10H-phenoxazine (1.35 g, 7.39 mmol) and NaO^{t}Bu (860 mg, 8.99 mmol) in toluene (50 mL) was degassed (N₂ bubbling, 15 min). Tris(dibenzylideneacetone)dipalladium(0) (29 mg, 0.032 mmol) and 1,1′-bis(diphenylphosphino)ferrocene (36 mg, 0.064 mmol) were added and the mixture was heated to reflux (24 h). The mixture was allowed to cool to room temperature and then filtered through silica gel washing with EtOAc/CH₂Cl₂ (15:85). The filtrate was concentrated to give a solid residue. The residue was purified by silica gel column chromatography (EtOAc/petrol/CH₂Cl₂ 3:97:0 then 6:94:0 then 6:80:20 then 6:60:40) to give 10-(4-(6-(10H-phenoxazin-10-yl)-4-phenylquinolin-2-yl)phenyl)-10H-phenoxazine 19 (900 mg, 44%) as colourless solid. A portion of this material was further purified by, firstly, recrystallisation (toluene/petrol) and, secondly, by (tube furnace) sublimation (285° C., 10⁻⁶ mBar): m.p. 298-302° C. (DSC). ¹H NMR (CDCl₃, 400 MHz) δ 5.97 (dd, J 1.5, 7.9 Hz, 2H), 6.06 (dd, J 1.6, 7.8 Hz, 2H), 6.57-6.74 (m, 12H), 7.48-7.60 (m, 7H), 7.72 (dd, J 2.3, 8.9 Hz, 1H), 7.94 (s, 1H), 7.98 (d, J 2.2 Hz, 1H), 8.42-8.47 (m, 2H), 8.50 (d, J 8.9 Hz, 1H); ¹³C NMR (CDCl₃, 100 MHz) δ 113.22, 113.34, 115.52, 115.59, 119.83, 121.48, 121.58, 123.26, 123.28, 127.28, 128.10, 128.90, 128.96, 129.44, 130.35, 131.41, 132.12, 133.75, 134.16, 134.18, 136.80, 137.47, 139.52, 140.34, 143.95, 148.36, 149.61, 156.90; HRMS (APCI) m/z 643.2255 C₄₅H₂₉O₂N₃ [M]^{+·} requires 643.2254.

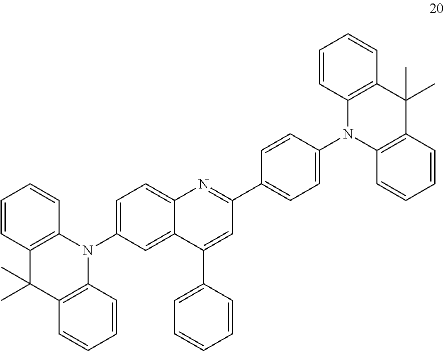 [SMILES: CC1(C)c2ccccc2N(c2ccc(-c3cc(-c4ccccc4)c4cc(N5c6ccccc6C(C)(C)c6ccccc65)ccc4n3)cc2)c2ccccc21]

### 10-(4-(6-(9,9-dimethylacridin-10(9H)-yl)-4-phenylquinolin-2-yl)phenyl)-9,9-dimethyl-9,10-dihydroacridine 20

A mixture of 6-bromo-2-(4-bromophenyl)-4-phenylquinoline (635 mg, 1.46 mmol), 9,9-dimethyl-9,10-dihydroacridine (670 mg, 3.21 mmol) and NaO^{t}Bu (390 mg, 4.08 mmol) in toluene (30 mL) was degassed (N₂ bubbling, 15 min). Tris(dibenzylideneacetone)dipalladium(0) (31 mg, 0.015 mmol) and 1,1′-bis(diphenylphosphino)ferrocene (16 mg, 0.03 mmol) were added and the mixture was heated to reflux (24 h). The mixture was allowed to cool to room temperature and then filtered through silica gel washing with EtOAc/CH₂Cl₂ (15:85). The filtrate was concentrated to give a solid residue. The residue was purified by silica gel column chromatography (EtOAc/petrol/CH₂Cl₂ 5:95:0 then 5:85:10 then 5:80:15) to give 10-(4-(6-(9,9-dimethylacridin-10(9H)-yl)-4-phenylquinolin-2-yl)phenyl)-9,9-dimethyl-9,10-dihydroacridine 20 (900 mg, 89%) as colourless solid. A portion of this material was further purified by, firstly, recrystallisation (EtOAc/petrol) and, secondly, by (tube furnace) sublimation (280° C., 10⁻⁶ mBar): m.p. 295-303° C. (DSC). ¹H NMR (CDCl₃, 400 MHz) δ 1.72 (s, 6H), 1.75 (s, 6H), 6.32 (dd, J 1.8, 7.4 Hz, 2H), 6.43 (dd, J 1.8, 8.1 Hz, 2H), 6.93-7.05 (m, 8H), 7.45-7.62 (m, 11H), 7.71 (dd, J 2.2, 8.8 Hz, 1H), 7.99-8.02 (m, 2H), 8.48-8.52 (m, 2H), 8.56 (d, J 8.8 Hz, 1H); ¹³C NMR (CDCl₃, 100 MHz) δ 31.34, 31.42, 35.98, 36.00, 114.04, 114.15, 119.94, 120.67, 120.75, 125.30, 125.42, 126.39, 126.45, 127.29, 128.12, 128.76, 128.88, 129.47, 130.06, 130.21, 131.91, 133.07, 133.07, 133.42137.63, 139.18, 139.39, 140.74, 140.77, 142.61, 148.37, 149.66, 157.02; HRMS (APCI) m/z 695.3294 C₅₁H₄₁N₃ [M]^{+·} requires 695.3295.

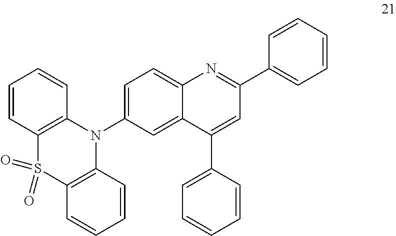 [SMILES: O=S1(=O)c2ccccc2N(c2ccc3nc(-c4ccccc4)cc(-c4ccccc4)c3c2)c2ccccc21]

### 10-(2,4-diphenylquinolin-6-yl)-10H-phenothiazine 5,5-dioxide 21

A mixture of 6-bromo-2,4-diphenylquinoline (800 g, 2.22 mmol), 10H-phenothiazine (510 mg, 2.55 mmol) and NaO^{t}Bu (300 mg, 3.11 mmol) in toluene (30 mL) was degassed (N₂ bubbling, 15 min). Tris(dibenzylideneacetone)dipalladium(0) (20 mg, 0.022 mmol) and 1,1′-bis(diphenylphosphino)ferrocene (25 mg, 0.044 mmol) were added and the mixture was heated to reflux (24 h). The mixture was allowed to cool to room temperature and then filtered through silica gel washing with EtOAc/CH₂Cl₂/petrol (8:30:62). The filtrate was concentrated to give a solid residue. The residue was purified by silica gel column chromatography (EtOAc/petrol 3:97 then 4:96) to give, presumably, 10-(2,4-diphenylquinolin-6-yl)-10H-phenothiazine (990 mg, 93%) as colourless solid. ¹H NMR (CDCl₃, 400 MHz) δ 6.24-6.31(m, 2H), 6.81-6.89 (m, 4H), 7.03-7.08 (m, 2H), 7.46-7.60 (m, 8H), 7.73 (dd, J 2.3, 8.9 Hz, 1H), 7.89 (s, 1H), 7.96 (d, J 2.2 Hz, 1H), 8.21-8.25 (m, 2H), 8.48 (d, J 8.9 Hz, 1H). Next, to 10-(2,4-diphenylquinolin-6-yl)-10H-phenothiazine (900 mg, 1.88 mmol) in CH₂Cl₂ (30 mL) was added 3-chloroperoxybenzoic acid (970 mg, 4.33 mmol). After 3 hours the mixture washed with saturated aqueous NaHCO₃, then saturated aqueous NaCl, then dried (MgSO₄), filtered and then concentrated to give a residue. The residue was purified firstly, by silica gel column chromatography (EtOAc/petrol/toluene 8:92:0 then 8:46:46 then 8:0:92) to give 10-(2,4-diphenylquinolin-6-yl)-10H-phenothiazine 5,5-dioxide 21 (770 mg, 80%) as colourless solid. A portion of this material was further purified by, firstly, recrystallisation (toluene/petrol) and, secondly, by (tube furnace) distillation (245° C., 10⁻⁶ mBar): m.p. 142-152° C. (DSC). ¹H NMR (CDCl₃, 400 MHz) δ 6.65 (d, J 8.4 Hz, 2H), 7.23-7.28 (m, 2H), 7.38 (ddd, J 1.6, 7.2, 8.8 Hz, 2H), 7.44-7.62 (m, 8H), 7.67 (dd, J2.3, 8.8 Hz, 1H), 7.97 (s, 1H), 8.00 (d, J2.2 Hz, 1H), 8.18 (dd, J 1.5, 7.9 Hz, 2H), 8.24-8.28 (m, 2H), 8.57 (d, J 8.8 Hz, 1H); ¹³C NMR (CDCl₃, 100 MHz) δ 117.09, 120.42, 122.18, 122.58, 123.52, 127.26, 127.55, 127.69, 129.02, 129.08, 129.30, 129.98, 131.27, 132.86, 133.87, 136.39, 137.17, 138.98, 140.78, 148.67, 149.69, 158.63; HRMS (APCI) m/z 511.1460 C₃₃H₂₃O₂N₂S [M+H]^{+*} requires 511.1475.

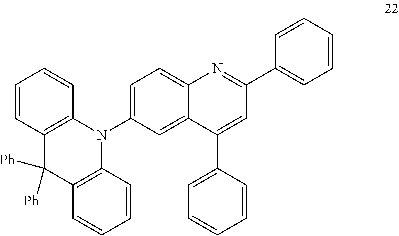 [SMILES: c1ccc(-c2cc(-c3ccccc3)c3cc(N4c5ccccc5C(c5ccccc5)(c5ccccc5)c5ccccc54)ccc3n2)cc1]

### 10-(2,4-diphenylquinolin-6-yl)-9,9-diphenyl-9,10-dihydroacridine 22

A mixture of 6-bromo-2,4-diphenylquinoline (1.05 g, 2.92 mmol), 9,9-diphenyl-9,10-dihydroacridine (1.12 g, 3.35 mmol) and NaO^{t}Bu (390 mg, 4.08 mmol) in toluene (30 mL) was degassed (N₂ bubbling, 15 min). Tris(dibenzylideneacetone)dipalladium(0) (26 mg, 0.029 mmol) and 1,1′-bis(diphenylphosphino)ferrocene (32 mg, 0.058 mmol) were added and the mixture was heated to reflux (3 h). The mixture was allowed to cool to room temperature and then filtered through silica gel washing with EtOAc/CH₂Cl₂/petrol (10:30:60). The filtrate was concentrated to give a solid residue. The residue was purified by silica gel column chromatography (EtOAc/petrol/toluene 0:50:50 then 0:20:80 then 2:20:80) to give 10-(2,4-diphenylquinolin-6-yl)-9,9-diphenyl-9,10-dihydroacridine 22 (1.54 g, 86%) as colourless solid. A portion of this material was further purified by, firstly, recrystallisation (toluene/petrol) and, secondly, by (tube furnace) distillation (275° C., 10⁻⁶ mBar): m.p. 255-262° C. (DSC). ¹H NMR (CDCl₃, 400 MHz) δ 6.48 (dd, J 0.9, 8.3 Hz, 2H), 6.87-6.95 (m, 4H), 6.99-7.08 (m, 6H), 7.20-7.28 (m, 6H), 7.43 (dd, J 2.2, 9.2 Hz, 1H), 7.47-7.60 (m, 8H), 7.73 (d, J 2.2 Hz, 1H), 7.90 (s, 1H), 8.22-8.26 (m, 2H), 8.43 (d, J 8.8 Hz, 1H); ¹³C NMR (CDCl₃, 100 MHz) δ 56.74, 113.99, 119.79, 120.30, 126.27, 126.84, 126.87, 127.59, 127.62, 127.91, 128.52, 128.78, 128.92, 129.39, 129.60, 129.71, 130.01, 130.32, 132.72, 132.85, 137.68, 138.47, 139.36, 142.06, 146.19, 148.23, 149.26, 157.67; HRMS (APCI) m/z 613.2632 C₄₆H₃₃N₂ [M+H]^{+·} requires 613.2638.

### Example 2

### Theoretical Calculations

Density Functional Theory (DFT) calculations for compounds 17-20 using Gaussian 09 (B3LYP/6-31G*) showed localized HOMO and LUMO levels across the electron donating area and electron accepting core respectively. Compounds 17-20 display limited overlap between the HOMO and LUMO levels. The S₁-T₁ gap for compounds 17-20 were calculated with Gaussian 09 (TD B3LYP/6-31G*//B3LYP/6-31G*) and predicted to be less than 0.1 eV with predicted emissions at approximately 550 nm, 534 nm, 578 nm and 510 nm, respectively.

TABLE 1
Theoretical calculations for compounds 17-20

| | S₁ | T₁ | ΔS₁ − T₁ | λ |
|---|---|---|---|---|
| Compound | (eV) | (eV) | (eV) | (nm) |
| 17 | 2.24 | 2.23 | 0.01 | 552 |
| 18 | 2.32 | 2.32 | 0.01 | 534 |
| 19 | 2.15 | 2.13 | 0.02 | 578 |
| 20 | 2.43 | 2.42 | 0.01 | 510 |

### Example 3

### Spectroscopy

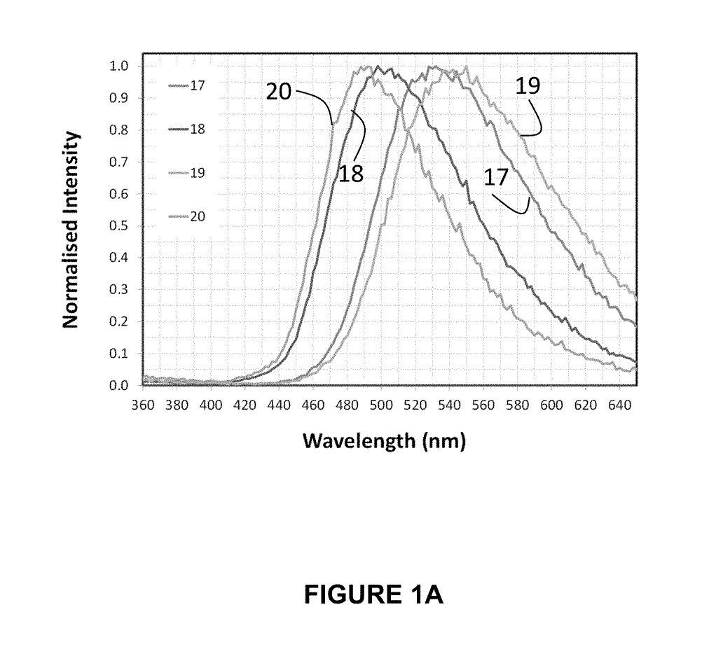

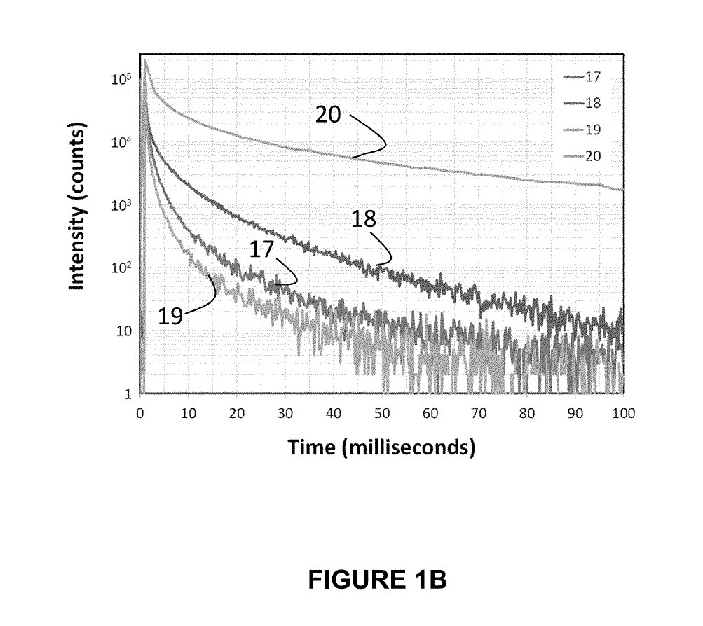

Steady-state and time-resolved photo-luminescence (PL) spectra of these compounds in encapsulated evaporated films (10% w/w co-evaporated 2,6-di(9H-carbazol-9-yl)pyridine, Pyd2Cz, matrix) were measured using an Edinburgh Instruments FLSP-900 spectrophotometer. The dominant highly Stokes-shifted PL band in the range 500-550 nm displays a strong solvatochromic effect which is consistent with this emission being associated with an excited-state intramolecular charge-transfer state, FIG. 1(a).

The presence of a very long-lived (millisecond) PL component, FIG. 1(b), together with the fact this long-lived emission is quenched in the presence of oxygen, suggests strongly the involvement of relatively long-lived triplet excitons, and is consistent with TADF enhancement of the fluorescence. A comparison of gated and ungated PL emission spectra indicates an efficient TADF process for these compounds.

TABLE 2
Physical and Spectroscopic Properties of Compounds 17-20

| | | | | | λ_{PL} | | |
|---|---|---|---|---|---|---|---|
| Com- | mp^{a} | HOMO^{b} | LUMO^{c} | E_{g}^{d} | (max)^{e} | | τ^{g} |
| pound | (° C.) | (eV) | (eV) | (eV) | (nm) | PLQY^{f} | (ms) |
| 17 | 173-178 | 5.42 | 2.68 | 2.74 | 530 | 0.85 | 2.3 |
| 18 | 224-227 | 5.44 | 2.61 | 2.83 | 500 | 0.68 | 6.5 |
| 19 | 296-304 | 5.54 | 2.91 | 2.63 | 542 | 0.95 | 1.6 |
| 20 | 292-298 | 5.56 | 2.68 | 2.88 | 490 | 0.46 | 48.3 |

^{a}Melting points were estimated from differential scanning calorimetry (DSC) analysis;
^{b}highest occupied molecular orbital (HOMO) energy levels were obtained from photoelectron spectroscopy in air (PESA);
^{c}lowest unoccupied molecular orbital (LUMO) energy levels were calculated by subtracting the band gap (E_{g}) from the HOMO level;
^{d}E_{g} measurements were obtained from the onset of the absorption band;
^{e}Photo-luminescence (PL) wavelength maxima;
^{f}PL quantum yield (PLQY) in Pyd2Cz films;
^{g}mean delayed-fluorescence lifetime obtained from fitting PL decay curves using a sum of exponentials.

### Example 4

### Device Fabrication and Testing

Commercial materials tris(4-carbazoyl-9-ylphenyl)amine (TcTa), 2,2′,2″-(1,3,5-benzinetriyl)-tris(1-phenyl-1-H-benzimidazole) (TPBi) and Pyd2Cz (2,6-di(9H-carbazol-9-yl)pyridine) were purchased from Lumtec and used as received.

OLED devices were fabricated on pre-patterned indium tin oxide (ITO) coated glass substrates (10 Ω/). Substrates were pre-cleaned with isopropanol and water and then treated with UV-ozone for 15 minutes at 25° C. immediately prior to use. Devices were fabricated via thermal evaporation in a vacuum chamber with a base pressure of around 5×10⁻⁶ Pa. The deposition rate for the organic layers was maintained at approximately 1 Å/s and 0.1 Å/s for the emitter.

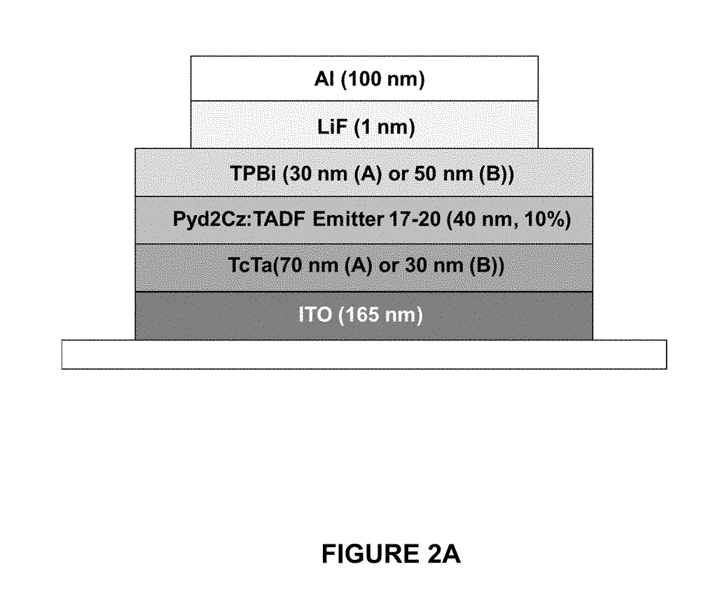

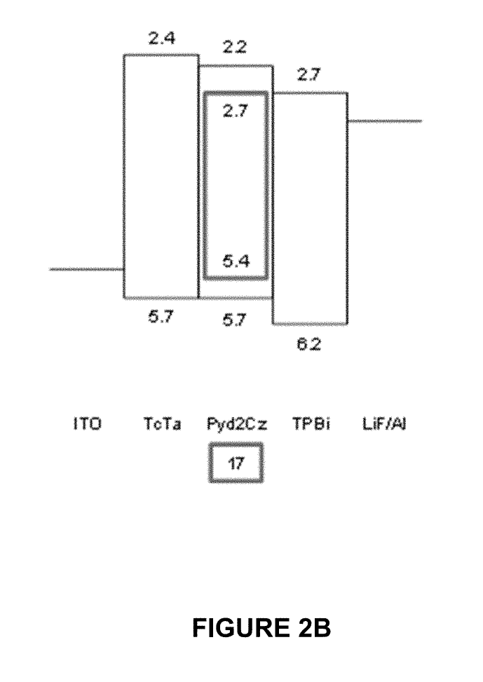

Devices were fabricated using a shadow mask to deposit successive layers of TcTa, Pyd2Cz doped with the test compound (17-20), and TPBi. After the organic layers were deposited the cathode layers, lithium fluoride (1 nm) and aluminium (100 nm), were deposited using a second shadow mask without breaking the vacuum. The device structure is shown in FIG. 2(a) with the relative energy levels for the materials, FIG. 2(b).

Compounds 17-20 were all initially tested in devices using 70 nm of TcTa, 40 nm total for the co-evaporated emissive layer (EML) and 30 nm of TPBi (Device A). Compound 19 was then used in a series of devices with different layer thicknesses, TcTa (30-70 nm), EML (20-40 nm) and TPBi (20-50 nm). For compound 19, device efficiency was found to be improved by using a thinner layer of TcTa (30 nm) and a thicker layer of TPBi (50 nm) (Device B). Compounds 17 and 18 were also prepared using Device B however no significant improvement was found for compounds 17 and 18 using this structure, results for these materials are reported for Device A. Compound 20 was only tested using Device A.

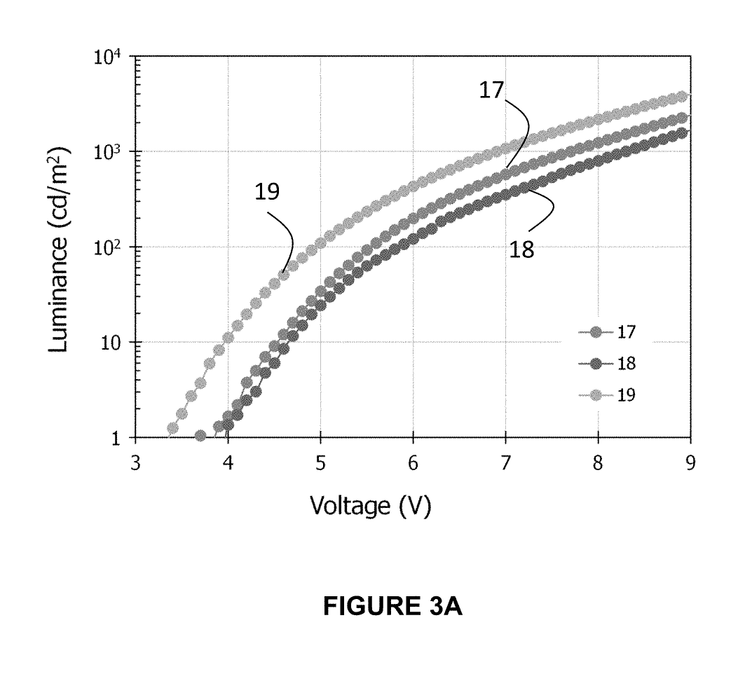

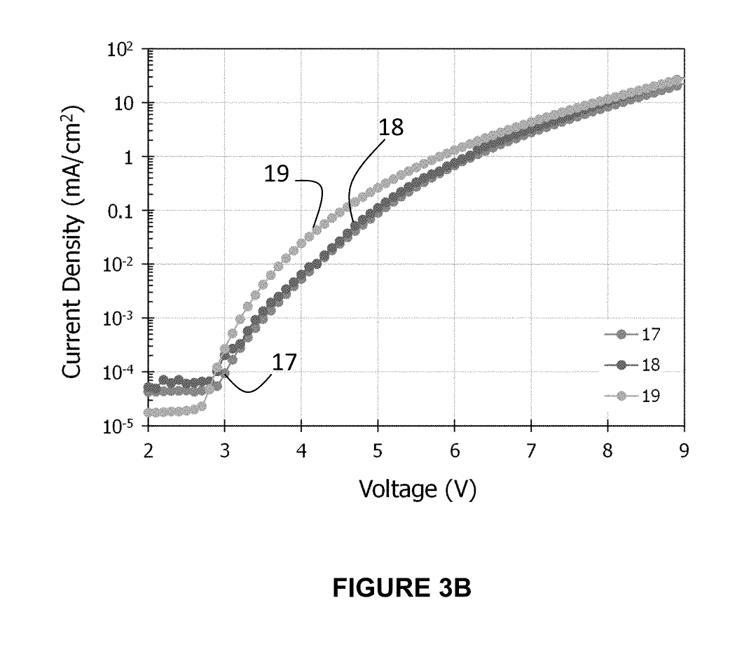

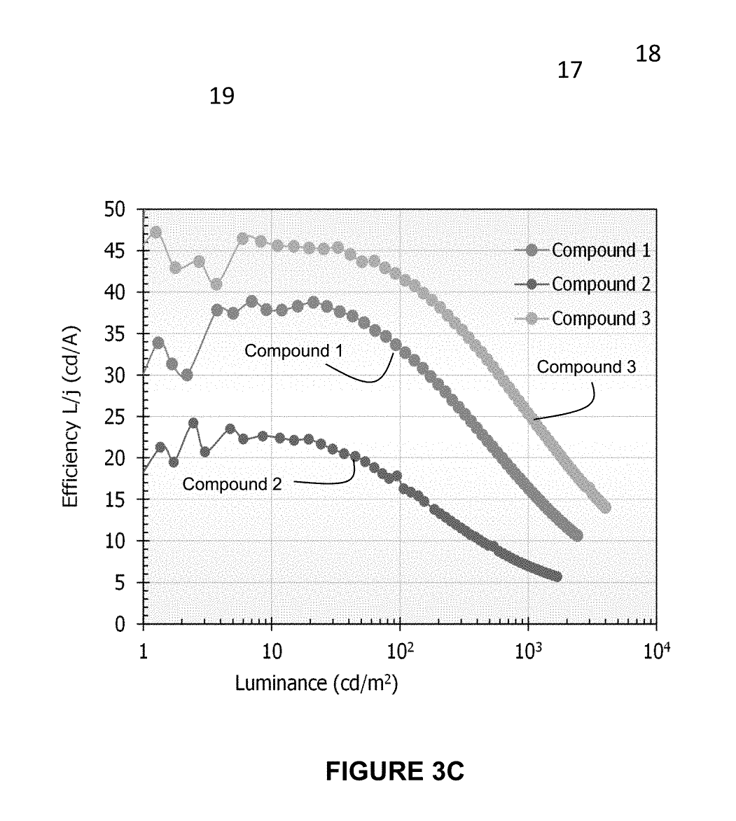

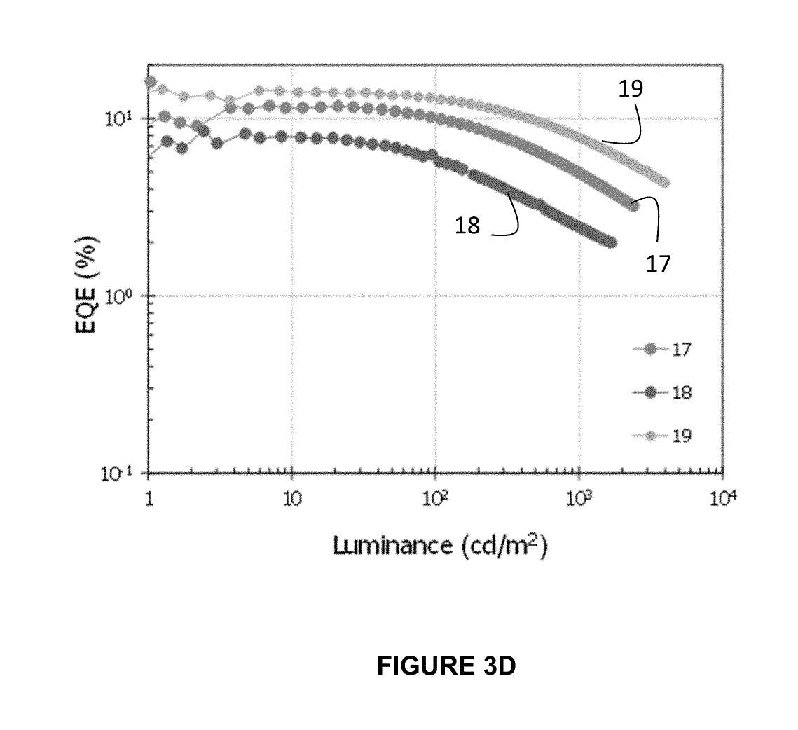

The voltage-current-luminance (VIL) characteristics of the OLEDs containing compounds 17-19 in the emitting layer are shown in FIG. 3 and the median performance of these devices at 1 mA/cm² is summarized in Table 3. Devices prepared with 17 and 19 have similar greenish yellow emissions with electroluminescence (EL) maxima at 533 and 548 nm. The device prepared with compound 18 exhibited a blue-green emission with an EL maximum at 508 nm.

Compound 19 with the symmetrical electron-donating groups had and maintaining an EQE of 7.8% at 1000 cd/m². Compound 17 had a peak EQE of 11.8% at low luminance and 5.0% at 1000 cd/m². Compound 16 had a lower peak efficiency of 7.8%. All three results are consistent with triplet involvement in the excited state and TADF as observed in the spectroscopy.

The device prepared with compound 20 had a sky blue emission at 489 nm and the CIE₁₉₃₁ coordinates for this device were (0.212, 0.228).

TABLE 3
Median VIL Performance at 1 mA/cm² for
Devices Prepared with Compounds 17-19

| | Volt- | Lumi- | | | | λ_{EL} | |
|---|---|---|---|---|---|---|---|
| Compound | age | nance | CIE₁₉₃₁ | CIE₁₉₃₁ | L/J | (max) | EQE |
| (Device) | (V) | (cd/m²) | x | y | (cd/A) | (nm) | (%) |
| 17 (A) | 6.1 | 258 | 0.351 | 0.577 | 25.8 | 533 | 8.1 |
| 18 (A) | 6.1 | 150 | 0.253 | 0.499 | 15.0 | 508 | 5.3 |
| 19 (B) | 5.7 | 328 | 0.407 | 0.557 | 32.8 | 548 | 10.8 |

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An electroluminescent device comprising a pair of electrodes forming an anode and a cathode, and one layer or multiple layers comprising a hole injection layer, emission layer and electron transporting layer, and wherein the emission layer comprises a light emitting compound represented by Formula (1): 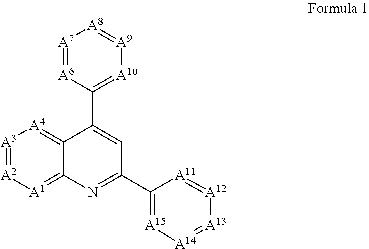
wherein
A¹, A², A³, A⁴, A⁶, A⁷, A⁸, A⁹, A¹⁰, A¹¹, A¹², A¹³, A¹⁴ and A¹⁵, are each independently selected from C—Y and N;
Each Y is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted monocyclic or polycyclic carbocyclic or heterocyclic, a moiety of Formula 2, and where two or more Y groups together form a fused aryl or heteroaryl group optionally substituted with a moiety of Formula 2, wherein the moiety of Formula 2 is represented by: 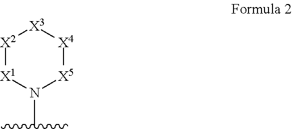
wherein X¹ to X⁵ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl, optionally substituted aryl or heteroaryl, an optionally substituted monocyclic or polycyclic heterocyclic, and two or more R⁴ and R⁵ groups may together form an optionally substituted monocyclic or polycyclic carbocyclic or heterocyclic;
R² and R³are each independently selected from hydrogen, C₁₋₁₀ alkyl, and monocyclic or polycyclic aryl and heteroaryl; and
wherein at least one of A¹, A², A³, A⁴, A⁶, A⁷, A⁸, A⁹, A¹⁰, A¹¹, A¹², A¹³, A¹⁴ and A¹⁵, is substituted with the moiety of Formula 2.

2. The device of claim 1, wherein each Y is independently selected from hydrogen, deuterium, halo, hydroxyl, nitro, cyano, thiol, amino, optionally substituted C₁₋₆alkyl, a moiety of Formula 2 as defined in claim 1, and where two or more Y groups together form a fused aryl or heteroaryl group optionally substituted with a moiety of Formula 2.

3. The device of claim 2, wherein each Y is independently selected from hydrogen, a moiety of Formula 2 as defined in claim 1, and where two or more Y groups together form a fused aryl or heteroaryl group optionally substituted with a moiety of Formula 2.

4-8. (canceled)

9. The device of claim 1, wherein:
A¹, A², A⁴, A⁶, A⁷, A⁸, A⁹, A¹⁰, A¹¹, A¹², A¹⁴ and A¹⁵, are each CH; and
A³ and A¹³ are each C—Y, wherein Y is a moiety of Formula 2 as defined in claim 1.

10. The device of claim 1, wherein:
A¹, A⁶, A⁷, A⁸, A⁹, A¹⁰, A¹¹, A¹², A¹³, A¹⁴ and A¹⁵, are each independently selected from C—Y and N;
A² and A⁴ are each CH;
A³ is C—Y, wherein Y is a moiety of Formula 2; and
wherein Y and Formula 2 are as defined in claim 1.

11. The device of claim 1, wherein:
A¹ is CH or N;
A² and A⁴ are each CH;
A³ is C—Y, wherein Y is a moiety of Formula 2 as defined in claim 1;
A⁶, A⁷, A⁸, A⁹, A¹⁰, A¹¹, A¹², A¹³, A¹⁴ and A¹⁵, are each independently selected from C—Y, wherein each Y is independently selected from hydrogen, a moiety of Formula 2, and where two or more Y groups together form a fused aryl or heteroaryl group optionally substituted with a moiety of Formula 2.

12. The device of claim 1, wherein the moiety of Formula 2 is a moiety of Formula 2(a): 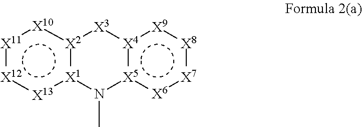
wherein X¹ to X¹³ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl, optionally substituted aryl or heteroaryl, and wherein the dotted circle indicates one or more optional double bonds; and
R² and R³are each independently selected from hydrogen and C₁₋₁₀alkyl.

13. The device of claim 1, wherein the moiety of Formula 2 is a moiety of Formula 2(a)(i): 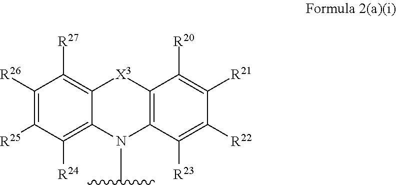
wherein X³ is selected from CR⁴R⁵, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl, and optionally substituted aryl or heteroaryl;
R² and R³are each independently selected from hydrogen and C₁₋₁₀ alkyl; and
R²⁰ to R²⁷ are each independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, and optionally substituted C₂₋₁₀ alkynyl.

14. The device of claim 1 selected from a compound of Formula 1(a): 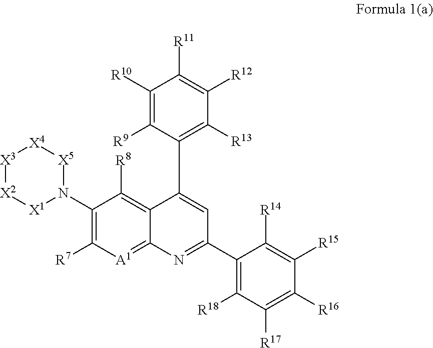
wherein
A¹ is CR⁶ or N;
X¹ to X⁵ are each independently selected from CR⁴, CR⁴R⁵, N, NR⁴, S, SiR⁴R⁵ and O, wherein each R⁴ and R⁵ are independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl, optionally substituted aryl or heteroaryl, an optionally substituted monocyclic or polycyclic heterocyclic, two or more R⁴ and R⁵ groups may together form an optionally substituted monocyclic or polycyclic carbocyclic or heterocyclic;
Each R² and R³ are independently selected from hydrogen, C₁₋₁₀alkyl, and monocyclic or polycyclic aryl and heteroaryl;
R⁶, R⁷ and R⁸, are each independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl; and
R⁹ to R¹⁸ are each independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl, optionally substituted aryl or heteroaryl, an optionally substituted monocyclic or polycyclic heterocyclic, a moiety of Formula 2 as defined in claim 1, and where two or more groups together form a fused aryl or heteroaryl group optionally substituted with a moiety of Formula 2.

15. The device of claim 1 selected from a compound of Formula 1(a)(i): 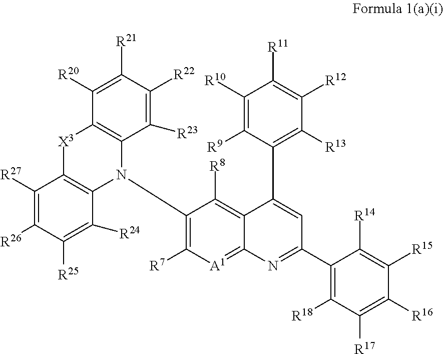
wherein
A¹ is CR⁶ or N,
X³ is selected from CR⁴R⁵, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl, optionally substituted aryl or heteroaryl;
R² and R³are each independently selected from hydrogen and C₁₋₁₀alkyl; and
R²⁹ to R²⁷ are ach independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl;
R⁶, R⁷ and R⁸, are each independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl;
R⁹ to R¹⁸ are each independently selected from hydrogen, deuterium, halo, CN, NO₂, C(O)R², OR², OS(O)₂R², NR²R³, SR², optionally substituted C₁₋₁₀alkyl, optionally substituted C₂₋₁₀ alkenyl, optionally substituted C₂₋₁₀ alkynyl, optionally substituted aryl or heteroaryl, an optionally substituted monocyclic or polycyclic heterocyclic, a moiety of Formula 2 as defined in claim 1, and where two or more groups together form a fused aryl or heteroaryl group optionally substituted with a moiety of Formula 2.

16. A thermally activated delayed fluorescence compound represented by Formula (1): 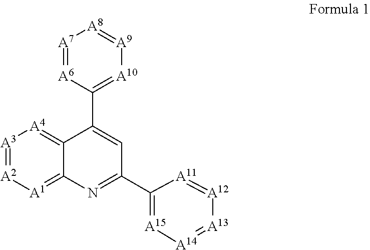
wherein
A¹, A², A³, A⁴, A⁶, A⁷, A⁸, A⁹, A¹⁰, A¹¹, A¹², A¹³, A¹⁴ and A¹⁵, are each independently selected from C—Y and N;
Each Y is independently selected from hydrogen, deuterium, halo, CN, NO₂, C(O)R², OR², OS(O)₂R², NR²R³, SR², optionally substituted C₁₋₁₀alkyl, optionally substituted C₂₋₁₀ alkenyl, optionally substituted C₂₋₁₀ alkynyl, optionally substituted monocyclic or polycyclic carbocyclic or heterocyclic, a moiety of Formula 2, and
where two or more Y groups together form a fused aryl or heteroaryl group optionally substituted with a moiety of Formula 2, wherein the moiety of Formula 2 is represented by: 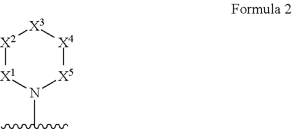
wherein X¹ to X⁵ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl, optionally substituted aryl or heteroaryl, an optionally substituted monocyclic or polycyclic heterocyclic, and two or more R⁴ and R⁵ groups may together form an optionally substituted monocyclic or polycyclic carbocyclic or heterocyclic;
wherein R⁴ and R⁵ cannot both be hydrogen;
R² and R³are each independently selected from hydrogen, C₁₋₁₀ alkyl, and monocyclic or polycyclic aryl and heteroaryl; and
wherein at least one of A¹, A², A³, A⁴, A⁶, A⁷, A⁸, A⁹, A¹⁰, A¹¹, A¹², A¹³, A¹⁴ and A¹⁵, is substituted with the moiety of Formula 2.

17. The compound of claim 16, wherein each Y is independently selected from hydrogen, a moiety of Formula 2 as defined in claim 16, and where two or more Y groups together form a fused aryl or heteroaryl group optionally substituted with a moiety of Formula 2.

18-21. (canceled)

22. The compound of claim 16, wherein:
A¹, A², A⁴, A⁶, A⁷, A⁸, A⁹, A¹⁰, A¹¹, A¹², A¹⁴ and A¹⁵, are each independently selected from C—Y;
A³ and A¹³ are each C—Y, wherein Y is a moiety of Formula 2; and
wherein Y and Formula 2 are as defined in claim 16.

23. The compound of claim 16, wherein:
A¹, A², A⁴, A⁶, A⁷, A⁸, A⁹, A¹⁰, A¹¹, A¹², A¹⁴ and A¹⁵, are each CH; and
A³ and A¹³ are each C—Y, wherein Y is a moiety of Formula 2 as defined in claim 16.

24. The compound of claim 16, wherein:
A¹, A⁶, A⁷, A⁸, A⁹, A¹⁰, A¹¹, A¹², A¹³, A¹⁴ and A¹⁵, are each independently selected from C—Y and N;
A² and A⁴ are each CH;
A³ is C—Y, wherein Y is a moiety of Formula 2; and
wherein Y and Formula 2 are as defined in claim 16.

25. The compound of claim 16, wherein:
A¹ is CH or N;
A² and A⁴ are each CH;
A³ is C—Y, wherein Y is a moiety of Formula 2 as defined in claim 16;
A⁶, A⁷, A⁸, A⁹, A¹⁰, A¹¹, A¹², A¹³, A¹⁴ and A¹⁵, are each independently selected from C—Y, wherein each Y is independently selected from hydrogen, a moiety of Formula 2, and where two or more Y groups together form a fused aryl or heteroaryl group optionally substituted with a moiety of Formula 2.

26. The compound of claim 16, wherein the moiety of Formula 2 is a moiety of Formula 2(a): 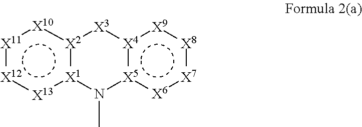
wherein X¹ to X¹³ are each independently selected from C, CR⁴, CR⁴R⁵, N, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀alkyl, optionally substituted C₂₋₁₀alkenyl, optionally substituted C₂₋₁₀alkynyl, optionally substituted aryl or heteroaryl, and wherein the dotted circle indicates one or more optional double bonds; and
R² and R³are each independently selected from hydrogen and C₁₋₁₀alkyl.

27. The compound of claim 16, wherein the moiety of Formula 2 is a moiety of Formula 2(a)(i): 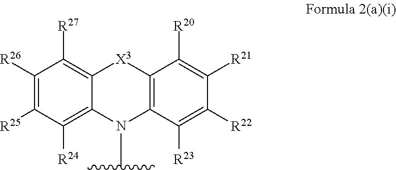
wherein X³ is selected from CR⁴R⁵, NR⁴, S, SO, SO₂, SiR⁴R⁵ and O; and each R⁴ and R⁵ are independently selected from hydrogen, deuterium, halo, CN, NO₂, C(O)R², OR², OS(O)₂R², NR²R³, SR², optionally substituted C₁₋₁₀ alkyl, optionally substituted C₂₋₁₀ alkenyl, optionally substituted C₂₋₁₀ alkynyl, and optionally substituted aryl or heteroaryl;
R² and R³are each independently selected from hydrogen and C₁₋₁₀ alkyl; and
R²⁰ to R²⁷ are each independently selected from hydrogen, deuterium, halo, CN, NO₂, C(O)R², OR², OS(O)₂R², NR²R³, SR², optionally substituted C₁₋₁₀alkyl, optionally substituted C₂₋₁₀ alkenyl, and optionally substituted C₂₋₁₀ alkynyl.

28. An emission material or host material comprising the compound of Formula 1 according to claim 16.

29-30. (canceled)

31. An organic electroluminescent device comprising an anode, a cathode and one or more layers arranged between the anode and the cathode, wherein at least one layer comprises an emission material or host material of claim 28.

32-39. (canceled)
